(19) United States
(12) Patent Application Publication
(10) Pub. No.: US 2026/0204427 A1
(43) Pub. Date: Jul. 16, 2026

(54) SEMANTIC LINKING OF TEXT TO STANDARDIZED MEDICAL CODES USING EMBEDDING-BASED SIMILARITY SEARCH

(71) Applicant: Oracle International Corporation, Redwood Shores, CA (US)

(72) Inventor: Yuanxu Wu, Sunnyvale, CA (US)

(73) Assignee: Oracle International Corporation, Redwood Shores, CA (US)

(21) Appl. No.: 19/447,835

(22) Filed: Jan. 13, 2026

Related U.S. Application Data
(60) Provisional application No. 63/745,016, filed on Jan. 14, 2025.

(51) Int. Cl. G16H 50/70; G06F 16/22; G06F 16/242; G06F 16/248

(52) U.S. Cl. CPC G16H 50/70; G06F 16/2237; G06F 16/243; G06F 16/248

(57) ABSTRACT

Unstructured clinical text, such as queries and extracted entities, is semantically normalized to standardized medical codings and schemas using embedding-based similarity search. Upon receiving clinical language input through an application programming interface, a semantic embedding model generates high-dimensional representations of the text. A hybrid search combining vector similarity and keyword matching is performed against a pre-indexed database of medical terminology and semantic object schemas. For broad queries, a ranked list of candidate codings and schemas is provided; for structured inputs containing pre-identified entities, each entity is linked to a ranked set of relevant concepts, with searches filtered by entity type and coding system. These approaches deliver accurate, context-aware mapping of clinical terms to standard vocabularies, enabling downstream clinical analytics, decision support, and data interoperability with high scalability and minimal latency.

### CROSS-REFERENCE TO RELATED APPLICATION

The present application is a non-provisional application of and claims the benefit and priority under 35 U.S.C. 119 (e) of U.S. Provisional Application No. 63/745,016, filed on Jan. 14, 2025, the entire contents of which is incorporated herein by reference in its entirety for all purposes.

### FIELD

The present disclosure relates generally to clinical data processing. More specifically, the present disclosure relates to linking natural language text, such as terms within a user query or a clinical note, to concepts within standardized medical coding systems, and exposes this functionality through a scalable micro-service architecture and application programming interfaces (APIs).

### BACKGROUND

The healthcare industry generates a vast and ever-increasing volume of data, a significant portion of which is unstructured or semi-structured text. This data, found in electronic health records (EHRs), clinical notes, lab reports, patient communications, and discharge summaries, contains a wealth of information critical for patient care, clinical research, and healthcare administration. However, the inherent variability and ambiguity of natural language present substantial challenges for automated processing and analysis. Medical concepts can be expressed in myriad ways, including formal medical terminology, common abbreviations, acronyms, colloquialisms, and even misspellings. For instance, a single clinical concept such as a “urinary tract infection” may be recorded as “UTI”, “bladder infection”, “acute cystitis”, or other variations, making it difficult for computer systems to recognize these as semantically equivalent.

To address this challenge and promote semantic interoperability, the medical community has developed a number of standardized coding systems. These systems provide a structured, controlled vocabulary for representing clinical information in an unambiguous and computable format. Key examples of such systems include: SNOMED CT (Systematized Nomenclature of Medicine-Clinical Terms), which is a comprehensive, multilingual clinical terminology designed to capture and represent detailed clinical information consistently and accurately; ICD-10-CM (International Classification of Diseases, Tenth Revision, Clinical Modification), which is used primarily for diagnosing and classifying diseases, symptoms, and injuries for billing and statistical purposes; LOINC (Logical Observation Identifiers Names and Codes), which is a standard for identifying medical laboratory observations, clinical tests, and measurements; and RxNorm, which provides normalized names for clinical drugs and links its names to many of the drug vocabularies commonly used in pharmacy management and drug interaction software.

The process of mapping the unstructured, free-text expression of a medical concept to its corresponding formal code within one of these standardized systems is known as concept linking, entity linking, or normalization. This normalization is a foundational step for unlocking the value of clinical data and enabling a host of advanced downstream applications. Without effective normalization, the utility of automated systems is severely limited. Therefore, there exists a significant and unmet need in the art for a more advanced, robust, and scalable solution for linking medical entities to standardized codes.

### BRIEF SUMMARY

Techniques are disclosed herein (e.g., a computer implemented method, a system, non-transitory computer-readable medium storing code or instructions executable by one or more processors) for linking natural language text, such as terms within a user query or a clinical note, to concepts within standardized medical coding systems, and exposes this functionality through a scalable micro-service architecture and APIs.

In various embodiments, a computer-implemented method is provided for that comprises: receiving, at an application programming interface endpoint, a request comprising an unstructured text query; generating, using a semantic embedding model and based on the unstructured text query, a query vector representing a semantic meaning of the unstructured text query; executing, using the query vector, a hybrid search against a pre-indexed vector database, wherein: the pre-indexed vector database comprises vector representations for medical codings and semantic object schemas, and the hybrid search combines a vector similarity search with a keyword search to retrieve a list of most relevant candidate codings and a list of most relevant candidate schemas; and providing, via the application programming interface, a structured response comprising a ranked list of the most relevant candidate codings and a ranked list of the most relevant candidate schemas.

In some embodiments, the computer-implemented method further comprises prior to generating the query vector, preprocessing the unstructured text query by performing key phrase extraction to identify one or more clinical terms in the unstructured text query, wherein the query vector is generated using the semantic embedding model and based on the one or more clinical terms.

In some embodiments, the vector similarity search is a k-Nearest Neighbors (kNN) vector similarity search that is executed based on the query vector to identify a first set of results, the keyword search is executed based on the query vector to identify a second set of results, and the first and second sets of results are combined based on a weight for the vector similarity search and a weight for the key word search to retrieve the list of most relevant candidate codings and the list of most relevant candidate schemas.

In some embodiments, the structured response is configured for consumption by a downstream schema synthesizer configured to construct a machine-readable representation of the unstructured text query.

In some embodiments, the request further comprises a search configuration, and wherein executing the hybrid search is governed by one or more parameters in the search configuration, the one or more parameters comprising a search size, a score threshold, a weight for the vector similarity search, a weight for the key word search, or any combination thereof.

In some embodiments, the computer-implemented method further comprises: generating, by one or more generative models based on the unstructured text query and the structured response, an execution plan that identifies one or more agents to execute one or more actions or operations responsive to the unstructured text query and the structured response; executing, by the one or more agents, the one or more actions or operations on one or more assets and/or a context and memory store, wherein the execution of the one or more actions or operations generates output data from the one or more assets and/or relevant context and memory information from the context and memory store; generating, by one or more generative models based on an output prompt, a response to the unstructured text query, wherein the output prompt comprises the output data and/or the relevant context and the memory information; and providing the response to the unstructured text query.

In some embodiments, the computer-implemented method further comprises: generating, by one or more generative models based on an output prompt, a response to the unstructured text query, wherein the output prompt comprises the unstructured text query and/or the ranked list of the most relevant candidate codings and the ranked list of the most relevant candidate schemas; and providing the response to the unstructured text query.

In various embodiments, a computer-implemented method is provided for that comprises: receiving, at an application programming interface endpoint, a structured request comprising one or more pre-identified text entities, each text entity of the one or more pre-identified text entities having an associated entity type; for each text entity, generating, using a semantic embedding model and based on the structured request, a query vector representing a semantic meaning of each entity; executing, using the query vector for each text entity, a hybrid search for each entity against a pre-indexed vector database, wherein: the pre-indexed vector database comprises vector representations for medical codings, the hybrid search combines a vector similarity search with a keyword search to retrieve a list of most relevant candidate codings and corresponding confidence scores, and the hybrid search is constrained by the entity type associated with the text entity; and providing, via the application programming interface, a structured response that associates each of the text entities with a ranked list of matched concepts based on the list of most relevant candidate codings and the corresponding confidence scores.

In some embodiments, the vector similarity search is a k-Nearest Neighbors (kNN) vector similarity search that is executed based on the query vector to identify a first set of results, the keyword search is executed based on the query vector to identify a second set of results, and the first and second sets of results are combined based on a weight for the vector similarity search and a weight for the key word search to retrieve the list of most relevant candidate codings.

In some embodiments, the one or more identified text entities in the structured request are generated by a Named Entity Recognition (NER) process applied to a natural language utterance.

In some embodiments, constraining the hybrid search based on the entity type associated with the text entity comprises searching only a subset of the pre-indexed vector database that corresponds to concepts associated with the entity type.

In some embodiments, the structured request further comprises a list of one or more target coding systems, and wherein the hybrid search for each text entity is further constrained to concepts belonging to the one or more target coding systems.

In some embodiments, the computer-implemented method further comprises: generating, by one or more generative models based on a natural language utterance, an execution plan that identifies one or more agents to execute one or more actions or operations responsive to the natural language utterance; executing, by the one or more agents, the one or more actions or operations on one or more assets and/or a context and memory store, wherein the execution of the one or more actions or operations generates output data from the one or more assets and/or relevant context and memory information from the context and memory store; generating, by one or more generative models based on an output prompt, a response to the natural language utterance, wherein the output prompt comprises: (i) the output data and/or the relevant context and the memory information, and (ii) the structured response that associates each of the text entities with the ranked list of matched concepts; and providing the response to the natural language utterance.

In some embodiments, the computer-implemented method further comprises: generating, by one or more generative models based on an output prompt, a response to a natural language utterance, wherein the output prompt comprises: (i) the natural language utterance, and (ii) the structured response that associates each of the text entities with a ranked list of matched concepts; and providing the response to the natural language utterance.

In some embodiments, the computer-implemented method further comprises: searching, using the structured response that associates each of the text entities with the ranked list of matched concepts, a semantic knowledge graph to identify one or more relationships between text entities; generating, by one or more generative models based on an output prompt, a response to a natural language utterance, wherein the output prompt comprises: (i) the natural language utterance, (ii) the structured response that associates each of the text entities with the ranked list of matched concepts, and the (iii) the one or more relationships between the text entities; and providing the response to the natural language utterance.

In various embodiments, a system is provided for that comprises: one or more processing systems; and one or more computer-readable media storing instructions which, when executed by the one or more processing systems, cause the system to perform operations comprising: receiving, at an application programming interface endpoint, a request comprising an unstructured text query or one or more pre-identified text entities; generating, using a semantic embedding model and based on the request, a query vector representing a semantic meaning of the unstructured text query or the one or more pre-identified text entities; executing, using the query vector, a hybrid search against a pre-indexed vector database, wherein: the pre-indexed vector database comprises vector representations for medical codings and semantic object schemas, and the hybrid search combines a vector similarity search with a keyword search to retrieve a list of most relevant candidate codings, a list of most relevant candidate schemas, or both; and providing, via the application programming interface, a structured response comprising: (i) a ranked list of the most relevant candidate codings and a ranked list of the most relevant candidate schemas, or (ii) a ranked list of matched concepts based on the list of most relevant candidate codings.

In some embodiments, the vector similarity search is a k-Nearest Neighbors (kNN) vector similarity search that is executed based on the query vector to identify a first set of results, the keyword search is executed based on the query vector to identify a second set of results, and the first and second sets of results are combined based on a weight for the vector similarity search and a weight for the key word search to retrieve the list of most relevant candidate codings, the list of most relevant candidate schemas, or both.

In some embodiments, the request further comprises a search configuration, and wherein executing the hybrid search is governed by one or more parameters in the search configuration, the one or more parameters comprising a search size, a score threshold, a weight for the vector similarity search, a weight for the key word search, or any combination thereof.

In some embodiments, the operations further comprise: generating, by one or more generative models based on a natural language utterance, the ranked list of the most relevant candidate codings, and the ranked list of the most relevant candidate schemas, an execution plan that identifies one or more agents to execute one or more actions or operations responsive to the natural language utterance; executing, by the one or more agents, the one or more actions or operations on one or more assets and/or a context and memory store, wherein the execution of the one or more actions or operations generates output data from the one or more assets and/or relevant context and memory information from the context and memory store; generating, by one or more generative models based on an output prompt, a response to the natural language utterance, wherein the output prompt comprises the output data and/or the relevant context and the memory information; and providing the response to the natural language utterance.

In some embodiments, the operations further comprise: generating, by one or more generative models based on an output prompt, a response to a natural language utterance, wherein the output prompt comprises: (i) the natural language utterance, and (ii) the structured response that associates each of the text entities with a ranked list of matched concepts; and providing the response to the natural language utterance.

Some embodiments include a system including one or more processors and one or more computer-readable media storing instructions which, when executed by the one or more processors, cause the system to perform part or all of the operations and/or methods disclosed herein.

Some embodiments include one or more non-transitory computer-readable media storing instructions which, when executed by one or more processors, cause a system to perform part or all of the operations and/or methods disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1 is an example of a healthcare environment that includes capabilities for providing various services to healthcare providers to facilitate care and management of their patient populations, in accordance with various embodiments.

FIG. 2 is an exemplary architecture for a generative model-based digital assistant, in accordance with various embodiments.

FIG. 3 is a simplified block diagram of a computing environment including a digital assistant that can execute an execution plan comprising actions and associated parameters for responding to an utterance from a user, in accordance with various embodiments.

FIG. 4 is a simplified block diagram of a computing environment for a Clinical Linking Service, in accordance with various embodiments.

FIG. 5 is an example flowchart depicting a method for performing medical entity linking using a search meaning candidates process, in accordance with various embodiments.

FIG. 6 is an example flowchart depicting a method for performing medical entity linking using a link to concept process, in accordance with various embodiments.

FIG. 7 is a block diagram illustrating one pattern for implementing a cloud infrastructure as a service system, in accordance with various embodiments.

FIG. 8 is a block diagram illustrating another pattern for implementing a cloud infrastructure as a service system, in accordance with various embodiments.

FIG. 9 is a block diagram illustrating another pattern for implementing a cloud infrastructure as a service system, in accordance with various embodiments.

FIG. 10 is a block diagram illustrating another pattern for implementing a cloud infrastructure as a service system, in accordance with various embodiments.

FIG. 11 is a block diagram illustrating an example computer system, in accordance with various embodiments.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### INTRODUCTION

In the field of clinical data processing, the automated, accurate, and efficient normalization of unstructured text to standardized medical concepts remains a significant challenge. Conventional systems designed to perform this entity or concept linking have traditionally relied on methods such as direct keyword matching, dictionary lookups, and complex, manually-curated rule-based engines. While these approaches have provided some utility, they suffer from numerous technical and practical limitations that impede their effectiveness, scalability, and adaptability, particularly in the context of modern, AI-driven healthcare applications.

A primary deficiency of these conventional systems is their brittleness and lack of semantic understanding. Rule-based and dictionary-based systems are often rigid; they can only identify terms that are explicitly pre-defined in their knowledge bases, e.g., keyword search is not effective to find related concepts. They struggle to handle the vast lexical variability of clinical language, including synonyms, acronyms (e.g., “UTI” for “urinary tract infection”), abbreviations, colloquialisms, and misspellings. Furthermore, these systems lack the ability to comprehend the underlying semantic context of a term. For example, they may fail to distinguish between different meanings of a word based on its surrounding text, leading to incorrect linkages. This requires constant and labor-intensive updates by domain experts to expand the dictionaries and refine the rules, a process that is both costly and unable to keep pace with the evolution of medical language and terminology.

Another significant limitation is the inflexibility and binary nature of their output. For example, prior linking services often provide only a single “best match” for a given term. This approach is fundamentally flawed as it fails to account for ambiguity. In many clinical scenarios, a term could plausibly map to several different, related concepts. By forcing a single output, the system denies downstream applications such as a clinical AI assistant the opportunity to apply further context-specific logic to disambiguate or to consider a ranked set of possibilities. This limitation severely hampers the capabilities of sophisticated AI agents that require a more nuanced understanding of clinical text.

Furthermore, conventional systems present substantial scalability and performance challenges, which manifest as an inefficient use of computing resources. As the size of medical terminologies like SNOMED CT (which contains hundreds of thousands of concepts) grows, the computational cost of searching through them using string-matching or executing an exhaustive set of rules for every query becomes prohibitive. This results in high latency, making such systems unsuitable for real-time or near-real-time applications, such as interactive AI assistants that must respond to a clinician's query instantly. The underlying architecture of these monolithic, rule-based systems is often not conducive to distributed computing, making it difficult to scale them horizontally to handle increasing query loads. This can lead to bottlenecks in the data processing pipeline, creating a drag on the entire healthcare IT infrastructure. Attempts to integrate such linking services into existing platforms are often hampered by rigid, non-standard APIs and an inability to customize the linking logic for specific use cases or data types.

In order to address these challenges and others, the present disclosure provides various embodiments for a Clinical Linking Service (CLS) that leverages an advanced, embedding-based similarity search architecture to deliver a highly accurate, scalable, and flexible solution for normalizing clinical text.

The various embodiments directly address the brittleness and lack of semantic understanding inherent in conventional systems. Instead of relying on pre-defined rules, the system utilizes a sophisticated machine learning model, such as an entity representation model, to generate high-dimensional vector embeddings for both the input text and the concepts within target medical coding systems. These embeddings capture the semantic meaning and context of the terms, not just their lexical form. This allows the system to recognize that terms like “heart attack” and “myocardial infarction” are semantically similar because their vector representations are close to each other in multi-dimensional space. This semantic approach inherently handles synonyms, acronyms, and novel phrasings far more robustly than any rule-based system.

To solve the problem of inflexible, binary outputs, the various embodiments are architected to provide a ranked list of candidate concepts rather than a single, forced match. This is exposed through a thoughtfully designed API, such as the searchMeaningCandidates endpoint, which returns a scored and ordered list of potential matches from various coding systems (e.g., SNOMED CT, LOINC, ICD-10-CM). This output provides flexibility for downstream applications, such as an AI agent or a schema synthesis tool, which can then use these candidates to make more intelligent, context-aware decisions. The API is also designed for customization, allowing a calling application to specify which coding systems to target and to fine-tune search parameters, thereby tailoring the service's behavior to a specific use case.

In a complementary manner, the various embodiments provide a more targeted approach for situations where entities within a text have already been identified, for instance, through a preceding Natural Language Processing (NLP) step such as Named Entity Recognition (NER). This is exposed through a second, more specific API, such as the linkToConcept endpoint, which is designed to receive a structured request containing one or more discrete text entities, each potentially associated with a specific type (e.g., “DIAGNOSIS,” “LAB_TEST,” “MEDICATION_NAME”). This function is optimized for a balance of high precision and recall, focusing on linking the specific, pre-identified entity to the most relevant and accurate codes within one or more designated coding systems. This targeted linking allows for the precise and structured annotation of clinical documents, enabling downstream processes to operate on a well-defined and normalized dataset

Lastly, the various embodiments described herein address the scalability and performance issues by implementing a technically superior architecture that optimizes the use of computing resources. The system is designed as a scalable micro-service that can be deployed and managed independently. At the core of this architecture is a dedicated vector similarity search engine, such as Qdrant or OpenSearch, which is specifically optimized for high-speed, low-latency nearest-neighbor searches on massive datasets of vectors. The concepts from the medical terminologies are pre-processed into vector embeddings and indexed within this engine. At query time, the system computes the vector for the input text and executes a highly efficient search against this pre-built index. This method is orders of magnitude faster and less computationally intensive than the brute-force searching or complex rule evaluation of conventional systems, resulting in significantly reduced latency, as evidenced by processing times measured in milliseconds. This architectural choice ensures that the system can handle a high volume of concurrent requests with minimal delay, making it perfectly suited for the demands of real-time clinical AI assistants.

Accordingly, the various embodiments provide for a computer-implemented method for linking clinical text to medical codes, the method comprising: receiving, at an application programming interface endpoint, an unstructured text query; generating a high-dimensional query vector representing the semantic meaning of the query using a semantic embedding model; executing a hybrid search against a pre-indexed vector database, wherein the database contains vector representations for both standardized medical codings and higher-level semantic object schemas; and providing, via the application programming interface, a structured response comprising a ranked list of candidate codings and a list of candidate schemas. The hybrid search combines vector similarity search with keyword search to retrieve the most relevant candidates, and the dual-component output is optimized for recall to enable downstream consumption by services such as a schema synthesizer, thereby providing a comprehensive, flexible, and semantically aware solution for interpreting broad clinical queries with reduced latency.

Furthermore, the various embodiments provide for an alternative computer-implemented method for linking specific clinical entities to medical codes, the method comprising: receiving, at an application programming interface endpoint, a structured request comprising one or more pre-identified text entities, each entity having an associated entity type; for each text entity, generating a high-dimensional entity vector using a semantic embedding model; executing a targeted and filtered search for each entity against a pre-indexed vector database, wherein the search is constrained by the entity's associated type and a specified list of target coding systems; and providing, via the application programming interface, a structured response that associates each of the identified text entities with its own ranked list of matched concepts and their corresponding confidence scores. This method is optimized for high precision and recall, thereby providing an accurate and efficient method for annotating specific clinical entities with standardized codes at minimal latency.

As used herein, when an action is “based on” something, this means the action is based at least in part on at least a part of the something.

As used herein, the terms “similarly”, “substantially,” “approximately” and “about” are defined as being largely but not necessarily wholly what is specified (and include wholly what is specified) as understood by one of ordinary skill in the art. In any disclosed embodiment, the term “similarly”, “substantially,” “approximately,” or “about” may be substituted with “within [a percentage] of” what is specified, where the percentage includes 0.1, 1, 5, and 10 percent.

### Overview

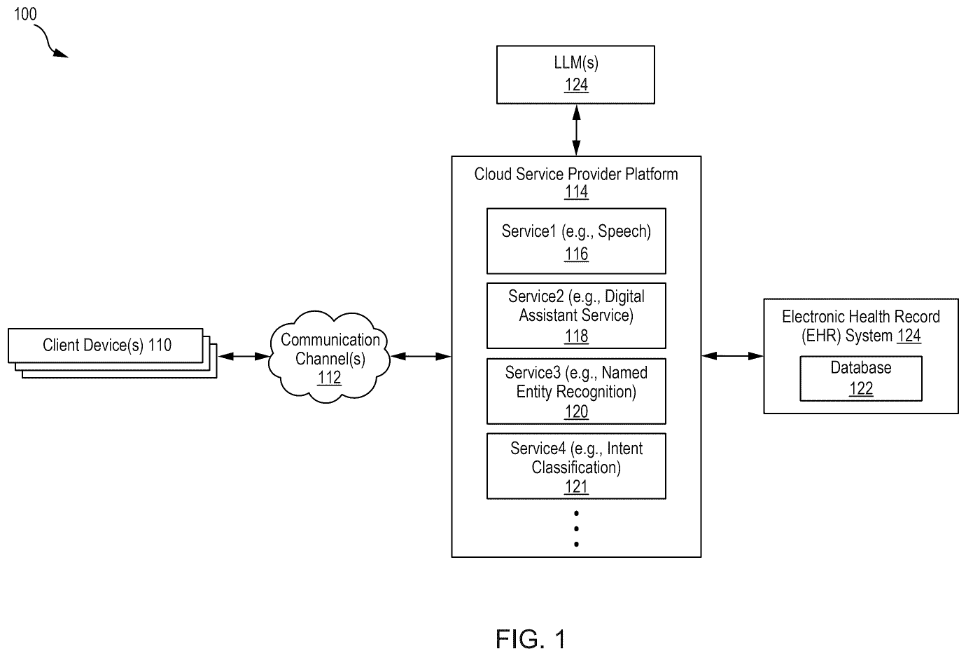

FIG. 1 is an example of a healthcare environment 100 that includes capabilities for providing various services to healthcare providers to facilitate care and management of their patient populations, according to certain embodiments. The term healthcare provider generally refers to healthcare practitioners and professionals including, but not limited to: physicians (e.g., general practitioners, specialists, surgeons, etc.); nurse professionals (e.g., nurse practitioners, physician assistants, nursing staff, registered nurses, licensed practical nurses, etc.); other professionals (e.g., pharmacists, therapists, technicians, technologists, pathologists, dietitians, nutritionists, emergency medical technicians, psychiatrists, psychologists, counselors, dentists, orthodontists, hygienists, etc.).

For the purpose of this disclosure, a healthcare environment may refer to the overall setting in which healthcare services are provided, such as hospitals, clinics, long-term care facilities, policy, procedure, and how care is delivered. A clinic environment may refer to the settings where patient care and clinical procedures are performed. In some embodiments, the terms healthcare environment and clinical environment may be used interchangeably.

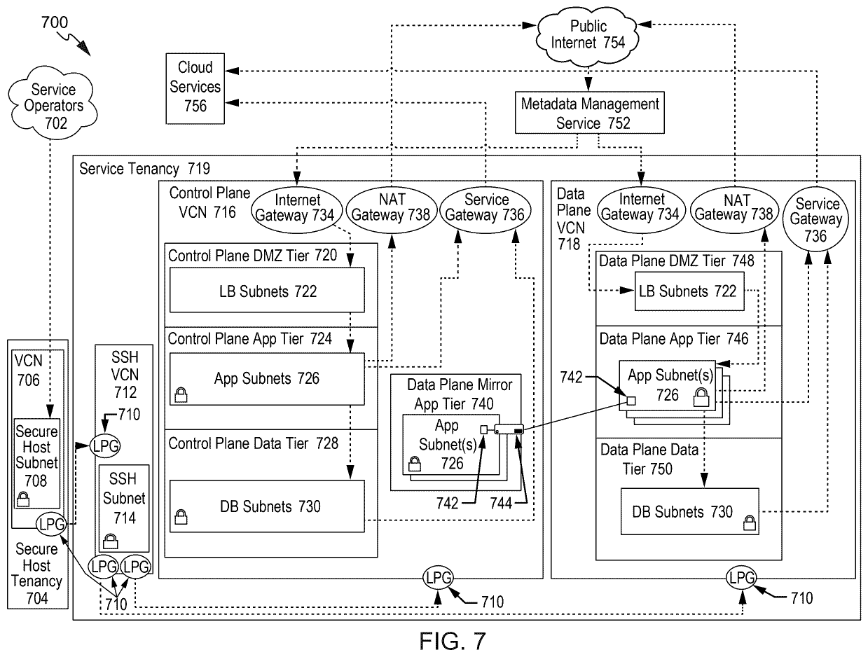

The healthcare environment 100 includes a cloud service provider platform 114 that includes capabilities for providing various services to subscribers (e.g., end-users) of the cloud service provider platform 114 (see, e.g., the IaasS architectures described with respect to FIGS. 7-11). The end-users (e.g., clinics such as doctors and nurses) may utilize the various services provided by the cloud service provider platform 114 to perform various functions involving the treatment, care, observation, and so on of patients. For instance, in the healthcare environment 100, the end-users can utilize the functionality provided by the services to view, edit, or manage a patient's electronic health record, perform administrative tasks such as scheduling appointments, manage patient populations, provide customer service to facilitate operation of the healthcare environment 100 and so on.

As shown in FIG. 1, the environment 100 includes one or more client devices 110 (hereinafter “client devices 110”), one or more communication channels 112 (hereinafter “communication channels 112”), a cloud service provider platform 114 (hereinafter “platform 114”), one or more databases 122 (hereinafter “databases 122”), and one or more generative models such as LLMs 124 (hereinafter “LLMs 124”). The platform 114, which can be included as part of a cloud infrastructure of a cloud service provider (e.g., Oracle Cloud Infrastructure or OCI), can be configured to communicate with, send data and information to, and receive data and information from the client devices 110 via the communication channels 112. Additionally, the platform 114 can be configured to access and/or call the databases 122 and the LLMs 124 to obtain and/or receive data and information from the databases 122 and the LLMs 124. Data and information received from the client devices 110, the databases 122, and the LLMs 124 can be used by the platform 114 to execute tasks and perform services. While FIG. 1 shows the databases 122 and the LLMs 124 as being separate from the platform 114, this is not intended to be limiting, and one or more of the databases 122 and/or one or more of the LLMs 124 can be included as part of the platform 114 and/or the cloud infrastructure in which the platform 114 is included. In some embodiments, the databases 122 may be part of an electronic health record (EHR) system 124, which may collect and store information relevant to the observation, care, treatment, and/or management of patients, and so on.

Each client device included in the client devices 110 can be any kind of electronic device that is capable of: executing applications; presenting information textually, graphically, and audibly such as via a display and a speaker; collecting information via one or more sensing elements such as image sensors, microphones, tactile sensors, touchscreen displays, and the like; connecting to a communication channel such as the communication channels 112 or a network such as a wireless network, wired network, a public network, a private network, and the like, to send and receive data and information; and/or storing data and information locally in one or more storage mediums of the electronic device and/or in one or more locations that are remote from the electronic device such as a cloud-based storage system, the platform 114, and/or the databases 122. Examples of electronic devices include, but are not limited to, mobile phones, desktop computers, portable computing devices, computers, workstations, laptop computers, tablet computers, and the like.

In some implementations, an application can be installed on, executed on, and/or accessed by a client device included in the client devices 110. The application and/or a user interface of the application can be utilized or interacted with (e.g., by an end user) to access, utilize, and/or interact with one or more services provided by the platform 114. The client device can be configured to receive multiple forms of input such as touch, text, voice, and the like, and the application can be configured to transform that input into one or more messages which can be transmitted or streamed to the platform 114 using one or more communication channels of the communication channels 112. Additionally, the client device can be configured to receive messages, data, and information from platform 114 using one or more communication channels of the communication channels 112, and the application can be configured to present and/or render the received messages, data, and information in one or more user interfaces of the application.

Each communication channel included in the communication channels 112 can be any kind of communication channel that is capable of facilitating communication and the transfer of data and/or information between one or more entities such as the client devices 110, the platform 114, the databases 122, and the LLMs 124. Examples of communication channels include, but are not limited to, public networks, private networks, the Internet, wireless networks, wired networks, local area networks, wide area networks, and the like. The communication channels 112 can be configured to facilitate data and/or information streaming between and among the one or more entities. In some implementations, data and/or information can be streamed using one or more messages and according to one or more protocols. Each of the one or more messages can be a variable-length message, and each communication channel included in the communication channel 112 can include a stream orchestration layer that can receive the variable length message in accordance with a predefined interface, such as an interface defined using an interface description language like AsyncAPI. Each of the variable-length messages can include context information that can be used to determine the route or routes for the variable-length message as well as a text or binary payload of arbitrary length. Each of the routes can be configured using a polyglot stream orchestration language that is agnostic to the details of the underlying implementation of the routing tasks and destinations.

Each database included in the databases 122 can be any kind of database that is capable of storing data and/or information and managing data and/or information. Data and/or information stored by each database can include data and/or information generated by, provided by, and/or otherwise obtained by the platform 114. Additionally, or alternatively, data and/or information stored and/or managed by each database can include data and/or information generated by, provided by, and/or otherwise obtained by other sources such as the client devices 110 and/or LLMs 124. One or more databases that are included in the databases 122 can be part of a platform for storing and managing healthcare information such as electronic health records for patients, electronic records of healthcare providers, and the like, and can store and manage electronic health records for patients of healthcare providers. An example platform is the Oracle Health Millenium Platform. Additionally, one or more databases included in the databases 122 can be provided by, managed by, and/or otherwise included as part of a cloud infrastructure of a cloud service provider (e.g., Oracle Cloud Infrastructure or OCI). Data and/or information stored and/or managed by the databases 122 can be accessed using one or more application programming interfaces (APIs) of the databases 122.

The platform 114 can be configured to include various capabilities and provide various services to subscribers (e.g., end users) of the various services. In some implementations, in the case of an end user or subscriber being a healthcare provider, the healthcare provider can utilize the various services to facilitate the observation, care, treatment, management, and so on of their patient populations. For example, a healthcare provider can utilize the functionality provided by the various services provided by the platform 114 to examine or treat a patient; view, edit, or manage a patient's electronic health record; perform administrative tasks such as scheduling appointments, managing patient populations, providing customer service to facilitate operation of a healthcare environment in which the healthcare provider practices, and so on.

In some implementations, the services provided by the platform 114 can include, but are not limited to, service 1 116 (e.g., a speech service), service 2 118 (e.g., a digital assistant service), service 3 120 (e.g., named entity recognition (NER) service), and service 4 121 (e.g., intent classification service). The speech service 116 can be configured to convert audio into text, such as a text transcript. For example, the speech service 116 can convert an audio recording of a conversation between a healthcare provider and their patient into a text transcript of the conversation. To convert audio into text, the speech service 116 can utilize one or more machine-learning models, such as an automatic speech recognition (ASR) model. In the case that the audio is streamed to the platform 114 in the form of messages (as described above) with each message including a portion of the audio (e.g., a one-second segment of the audio), in some implementations, the platform 114 and/or the speech service 116 can be configured to aggregate and combine all of the messages pertaining to the audio (e.g., all of the messages pertaining to a conversation) into audio data or an audio file prior to converting the audio data or audio file into text or a text transcript. In other implementations, the platform 114 and/or the speech service 116 can be configured to convert audio into text or a text transcript as the audio is received by the platform 114 and/or the speech service 116. The text or text transcript generated by the speech service 116 can be stored within the platform 114 and/or in another location, such as in one or more databases of the databases 122, where it can be accessed by the platform 114 and/or one or more other services of the platform 114 such as the digital assistant service 118 and/or the named entity recognition (NER) service 120. Additionally, or alternatively, the text or text transcript generated by the speech service 116 can be provided to one or more other services of the platform 114, such as the digital assistant service 118 and/or the NER service 120.

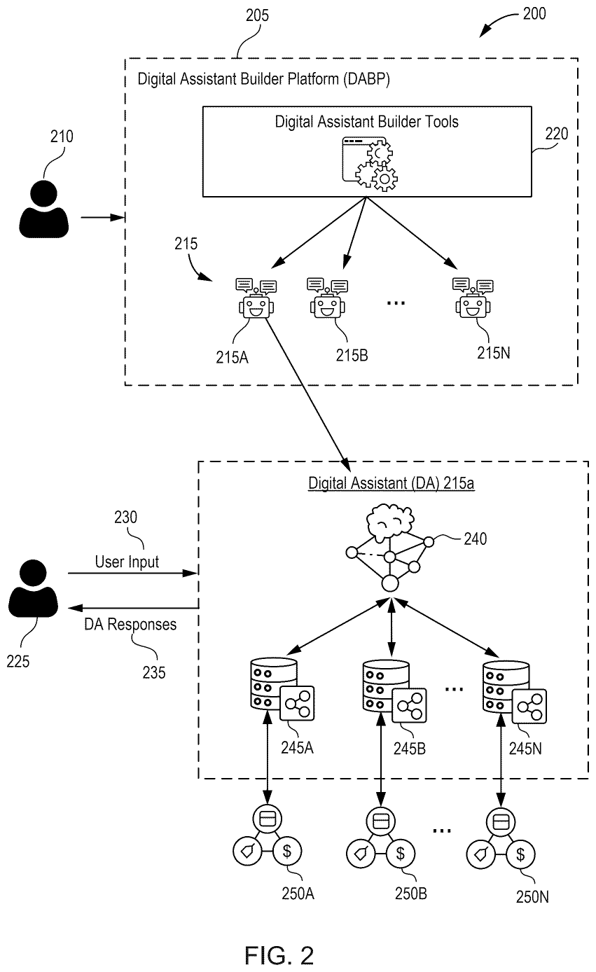

The digital assistant service 118 can be configured to serve as an artificial intelligence-driven (AI-driven) conversational-type interface for the platform 114 that can conduct conversations with end users (e.g., those using the client devices 110) and perform functions and/or tasks based on the information conveyed by and/or ascertained from those conversations and other sources (see description of FIG. 2 for additional detail). The digital assistant service 118 can be configured with and/or configured to access natural language understanding (NLU) capabilities such as natural language processing, named entity recognition, intent classification, and so on. In some implementations, the digital assistant service 118 can be skill-driven in which the digital assistant service 118 includes bots that each include one or more skills for conducting conversations and performing functions and/or tasks. In some implementations, the digital assistant service 118 can be generative model-based and agent-driven in which agent(s) coordinate with one or more generative models such as LLM(s) for conducting conversations and performing functions and/or tasks.

The digital assistant service 118 can be configured to initiate a dialog, drive a previously initiated dialog (e.g., by responding to a turn in the dialog), and/or otherwise participate in a conversation. In some implementations, the digital assistant service 118 can drive and/or participate in a dialog and/or conversation in response to events that have occurred at the client devices 110, the platform 114, the databases 122, the LLMs 124, and/or at the cloud infrastructure supporting the platform 114. In the case of a skill-driven digital assistant service 118, the events can be mapped to a particular skill and can trigger a flow for that skill. The flow can then generate response events/messages that can be used to render a user interface such as a user interface of a client application executing on the client devices 110. In the case of an LLM-based and agent-drive digital assistant service 118, the events can be mapped to a particular prompt or prompts to retrieve a result or results for the prompt or prompts, which can then be used to render the user interface. In some implementations, the digital assistant service 118 can drive and/or participate in a dialog and/or conversation in response to messages received from the client devices 110, the platform 114, the databases 122, the LLMs 124, and/or at the cloud infrastructure supporting the platform 114. In the case of a skill-driven digital assistant service 118, the messages can be routed to a particular skill, which, as described above, can generate response events/messages for rendering the user interface. In the case of an LLM-based and agent-driven digital assistant service 118, the metadata included in the messages can be used to generate and/or access a particular prompt or prompts to retrieve a result and/or results that can be used to render the user interface.

The named entity recognition (NER) service 120 can be configured to extract named entities in interest from natural language text. NER may be used in applications that are tasked with understanding the meaning of language text. In some embodiments, the NER service 120 may be part of the digital assistant service 118. For example, NER is commonly used in natural language processing applications to identify entities in natural language text. An entity could be a word or a string of words. Given a text input, which could be one or more words, one or more sentences, one or more paragraphs, etc., NER techniques are used to identify one or more entities from the input text and associate an entity type (may also referred to as category) with each entity. For example, an entity “John Doe” may be extracted from a piece of text and tagged or labeled with a “Person” category, entity “New York” may be tagged or labeled with a “Location” category, and the like.

Some entities may be hierarchical (referred to as hierarchical entities or nested entities). For a hierarchical entity, multiple sub-entities may be associated with an extracted named entity where the tagged entity types (or categories) are hierarchically related to each other. A named entity is thus labeled at multiple levels, each level adding further details about the entity to the previous level label. For example, in the sentence “order Tylenol oral tablets,” the named entity “Tylenol, tablets” may be tagged with a “medication” category (i.e., entity type) at a first hierarchical level (called level 1), and “tablets” may additionally be tagged with a “dose form” category at second hierarchical level (called level 2), where hierarchical level 2 is a sub-category (i.e., sub-entity type) of the hierarchical level 1 category (e.g., medication is a category at hierarchical level 1, and dose form is a sub-category of medication).

In certain embodiments, entities may also be categorized from a service perspective into several service categories/types, such as (1) built-in entities, (2) custom entities, (3) value list entities. The NER service 120 may include one or more machine learning models trained to label name entities with these types. Built-in entities are generic entities that can be used with a wide variety of bots. Examples of built-in entities include, without limitation, entities related to time, date, locations, addresses, numbers, email addresses, duration, recurring time periods, currencies, phone numbers, URLs, and the like.

Custom entities are used for more customized applications and may be handled by skill bots. For example, for a banking skill, an AccountType entity (part of the custom entities) may be defined by a banking skill bot designer that enables various banking transactions by checking the user input for keywords like checking, savings, and credit cards, etc. In some embodiments, a composite bag that identifies a group of related entities may be created for a particular business domain. For example, a composite bag for a pizza might include entities for type, size, crust, and extra toppings. If a user enters “I'd like a large pepperoni pizza with a gluten-free crust”, the skill could extract “large”, “pepperoni”, and “gluten-free” from that input and not need to prompt the user for those values individually. In some applications, a composite bag may help a skill bot to prompt for individual entity values when they are missing from the user input.

Value list entities are entities based on a list of predetermined values, like menu items that are output by a common response component. A customer can optimize the entity's ability to extract user input by defining synonyms. These can include abbreviations, slang terms, and common misspellings, etc. Synonym values are not case-sensitive. For example, “USA”, “United States”, and “America” may be part of a value list. A technique called elastic search can utilize the value list entities to perform a faster and efficient search on a gazetteer (i.e., a list of terms or words that are named entities) to recognize specific entities or terms in a domain the skill bot operates in to improve the accuracy of entity extraction in user inputs.

The intent classification/detection service 121 can be configured to map a user's utterance to a specific task/action or category of task/action that a chatbot can perform. The intent classification service may include an intent classifier that can be trained, using machine learning, LLM-based and/or rules-based training techniques, to determine a likelihood that an utterance is representative of a task that a particular skill bot is configured to perform. In some embodiments, the intent classifier may be trained on different classes, one class for each skill bot. For example, the utterance “I need to change my flight reservation” may be categorized under the intent “change reservation” class for airline.

Although not shown, the platform 114 can include other capabilities and services such as authentication services, management services, task management services, notification services, and the like. The various capabilities and services of the platform 114 can be implemented utilizing one or more computing resources and/or servers of the platform 114 and provided by the platform 114 by way of subscriptions. Additionally, or alternatively, while FIG. 1 shows the services of the platform 114 as being separate services, one or more of the services can be combined with other services and/or be considered to be a sub-service of another service. For example, the NER service 120 and/or the intent classification/detection 121 can be a sub-service or a service that is part of the digital assistant service 118.

### Digital Assistant and Knowledge Dialog

A bot (also referred to as an agent, chatbot, chatterbot, or talkbot), implemented as part of or as a digital assistant or AI assistant, is a computer program that can perform conversations with end users. The bot can generally respond to natural-language messages (e.g., questions or comments) through a messaging application that uses natural-language messages. Enterprises may use one or more bot systems to communicate with end users through a messaging application. The messaging application, which may be referred to as a channel, may be an end user preferred messaging application that the end user has already installed and familiar with. Thus, the end user does not need to download and install new applications in order to chat with the bot system. The messaging application may include, for example, over-the-top (OTT) messaging channels (such as Facebook Messenger, Facebook WhatsApp, WeChat, Line, Kik, Telegram, Talk, Skype, Slack, or SMS), virtual private assistants (such as Amazon Dot, Echo, or Show, Google Home, Apple HomePod, etc.), mobile, web, and cloud application extensions or plugins that extend native or hybrid/responsive mobile, web, or cloud applications with chat capabilities, or voice based input (such as devices or apps with interfaces that use Siri, Cortana, Google Voice, or other speech input for interaction).

In some examples, a bot system may be associated with a Uniform Resource Identifier (URI). The URI may identify the bot system using a string of characters. The URI may be used as a webhook for one or more messaging application systems. The URI may include, for example, a Uniform Resource Locator (URL) or a Uniform Resource Name (URN). The bot system may be designed to receive a message (e.g., a hypertext transfer protocol (HTTP) post call message) from a messaging application system. The HTTP post call message may be directed to the URI from the messaging application system. In some embodiments, the message may be different from a HTTP post call message. For example, the bot system may receive a message from a Short Message Service (SMS). While discussion herein may refer to communications that the bot system receives as a message, it should be understood that the message may be an HTTP post call message, a SMS message, or any other type of communication between two systems.

End users may interact with the bot system through a conversational interaction (sometimes referred to as a conversational user interface (UI)), just as interactions between people. In some cases, the interaction may include the end user saying “Hello” to the bot and the bot responding with a “Hi” and asking the end user how it can help. In some cases, the interaction may also be a transactional interaction with, for example, a banking bot, such as transferring money from one account to another; an informational interaction with, for example, a HR bot, such as checking for vacation balance; or an interaction with, for example, a retail bot, such as discussing returning purchased goods or seeking technical support.

In some embodiments, the bot system may intelligently handle end user interactions without interaction with an administrator or developer of the bot system. For example, an end user may send one or more messages to the bot system in order to achieve a desired goal. A message may include certain content, such as text, emojis, audio, image, video, or other method of conveying a message. In some embodiments, the bot system may convert the content into a standardized form (e.g., a representational state transfer (REST) or API call against enterprise services with the proper parameters) and generate a natural language response. The bot system may also prompt the end user for additional input parameters or request other additional information. In some embodiments, the bot system may also initiate communication with the end user, rather than passively responding to end user utterances. Described herein are various techniques for identifying an explicit invocation of a bot system and determining an input for the bot system being invoked. In certain embodiments, explicit invocation analysis is performed by a master bot based on detecting an invocation name in an utterance. In response to detection of the invocation name, the utterance may be refined or pre-processed for input to a bot that is identified to be associated with the invocation name and/or communication.

FIG. 2 is a simplified block diagram of an environment 200 incorporating a digital assistant system (also described herein as simply a digital assistant or in more specific terms with reference to implementation of agents as an agent assistant) according to certain embodiments. Environment 200 includes a digital assistant builder platform (DABP) 205 that enables users 210 to create and deploy digital assistant systems 215. For purposes of this disclosure, a digital assistant is an entity that helps users of the digital assistant accomplish various tasks through natural language conversations. The DABP and digital assistant can be implemented using software only (e.g., the digital assistant is a digital entity implemented using programs, code, or instructions executable by one or more processors), using hardware, or using a combination of hardware and software. In some instances, the environment 200 is part of an Infrastructure as a Service (IaaS) cloud service (as described below in detail with respect to FIGS. 7-11) and the DABP and digital assistant can be implemented as part of the IaaS by leveraging the scalable computing resources and storage capabilities provided by the IaaS provider to process and manage large volumes of data and complex computations. This setup allows the DABP and digital assistant to deliver real-time, responsive interactions while ensuring high availability, security, and performance scalability to meet varying demand levels. A digital assistant can be embodied or implemented in various physical systems or devices, such as in a computer, a mobile phone, a watch, an appliance, a vehicle, and the like. A digital assistant is also sometimes referred to as a chatbot system. Accordingly, for purposes of this disclosure, the terms digital assistant and chatbot system are interchangeable.

DABP 205 can be used to create one or more digital assistant systems (or DAs). For example, as illustrated in FIG. 2, user 210 representing a particular enterprise can use DABP 205 to create and deploy a digital assistant 215A for users of the particular enterprise. For example, DABP 205 can be used by a bank to create one or more digital assistants for use by the bank's customers, for example to change a 401k contribution, etc. The same DABP 205 platform can be used by multiple enterprises to create digital assistants. As another example, an owner of a restaurant, such as a pizza shop, may use DABP 205 to create and deploy digital assistant 215B that enables customers of the restaurant to order food (e.g., order pizza).

To create one or more digital assistant systems 215, the DABP 205 is equipped with a suite of tools 220, enabling the acquisition of machine learning models, agent creation, asset identification, and deployment of digital assistant systems within a service architecture for users via a computing platform such as a cloud computing platform described in detail with respect to FIGS. 7-11. In the specific context of this disclosure, the machine learning model(s) may be one or more generative models. A generative model is a machine learning model that is capable of generating new data instances based on the data used to train the model. A generative model may be referred to as a “generative artificial intelligence (AI) model.” Generative models learn the underlying distribution of the training data, enabling them to produce new instances of data that share properties with the original dataset. This capability makes them particularly useful in a variety of applications, including image and voice generation, text or code synthesis, and more sophisticated tasks like unsupervised learning, semi-supervised learning, and domain adaptation.

One type of generative model is a large language model (LLM). Large language models are designed to understand, generate, and interpret human language by processing extensive collections of data. The foundational architecture behind large language models is the transformer network, a type of neural network that excels in handling sequential data such as text. Unlike architectures, such as recurrent neural networks (RNNs) or long short-term memory networks (LSTMs), transformers do not process data in order. Instead, they leverage parallel processing to analyze entire text sequences simultaneously, significantly improving efficiency and reducing training times and inference latency times.

A mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

Transformers are composed of multiple layers containing a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to every other element is calculated using a scaled dot-product attention function that computes the attention scores by taking the dot product of the query vector with the key vectors, dividing each by the square root of the dimension of the key vectors to scale the scores, then applying a softmax function to obtain the weights for the value vectors. The scaled dot-product attention function is applied independently by each head in the multi-head self-attention mechanism. The outputs of these heads are then concatenated and linearly transformed, allowing the model to capture information from different representation subspaces.

Following the multi-head, self-attention mechanism is the position-wise, feed-forward network. This component comprises two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model's hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

Integrating these components within the transformer architecture facilitates the model's ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

Another type of generative model is a large multimodal model (LMM). A large multimodal model is an advanced machine learning model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for applications such as image captioning, text-to-image generation, image-to-text generation, visual question answering, and more, where understanding the relationship between different data types is crucial. By leveraging diverse datasets during training, large multimodal models learn to create coherent and contextually relevant outputs across various modalities, enhancing their utility in complex, real-world scenarios.

The architecture of large multimodal models combines elements from different neural network designs to handle diverse data types effectively. For example, convolutional neural networks (CNNs) are often used for processing visual data, while transformer networks handle textual data, enabling the model to extract and synthesize features from both images and text. This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type.

In at least some instances, the self-attention mechanism, a cornerstone of transformer networks, is integral to the functioning of large multimodal models. It enables the model to weigh the importance of different elements within an input sequence, regardless of their position, allowing it to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In large multimodal models, data preprocessing is a step that ensures the input data is in a suitable format for the model to process. This involves tasks such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is essential for the model to integrate information from various modalities and produce accurate, meaningful outputs.

Training large multimodal models involves optimizing their parameters through exposure to diverse datasets that include paired data from different modalities. This computationally intensive process often requires specialized hardware like GPUs or TPUs to manage the large volumes of data and the complexity of the model calculations. Techniques such as dropout and layer normalization are employed to improve model generalization and prevent overfitting. By iteratively adjusting the model's parameters, the training process enables the model to learn underlying patterns and relationships within the data, enhancing its ability to generate coherent and contextually relevant outputs across different modalities.

Evaluation and tuning of large multimodal models are conducted using various metrics tailored to the specific tasks they are designed to perform. For example, BLEU scores are used for text generation tasks, while accuracy is commonly applied for visual recognition tasks to assess performance. Tuning involves adjusting hyperparameters and refining training strategies based on evaluation results to enhance the model's effectiveness. This iterative process ensures that the model can perform a wide range of multimodal tasks with high accuracy and relevance, making it a versatile tool for applications requiring the integration of different types of data.

Large multimodal models represent a significant advancement in machine learning by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides large language models and large multimodal models belong to the broad category of generative models. For example, stochastic models directly incorporate randomness into their structure, making them inherently generative as they can produce a diverse set of outputs for a given input. Generative Adversarial Networks (GANs) learn to generate new data that is indistinguishable from the data they were trained on, using a dual-network architecture that involves a generative component. Variational Autoencoders (VAEs) are explicitly designed for generating new data points by learning a distribution of the input data and encode inputs into a latent space and generate outputs by sampling from this space, making them inherently generative. Sequence-to-sequence models are generative in nature when used with sampling strategies. Although this list of generative model types is not exhaustive, it illustrates the broad use of the term generative model beyond large language models.

In some instances, the tools 220 can be utilized to access pre-trained and/or fine-tuned generative models such as LLMs from data repositories or computing systems. The pre-trained LLMs serve as foundational elements, possessing extensive language understanding derived from vast datasets. This capability enables the models to generate coherent responses across various topics, facilitating transfer learning. Pre-trained models offer cost-effectiveness and flexibility, which allows for scalable improvements and continuous pre-training with new data, often establishing benchmarks in Natural Language Processing (NLP) tasks. Conversely, fine-tuned models are specifically trained for tasks or industries (e.g., plan creation utilizing the LLM's in-context learning capability, knowledge or information retrieval on behalf of an agent, response generation for human-like conversation, etc.), enhancing their performance on specific applications and enabling efficient learning from smaller, specialized datasets. Fine-tuning provides advantages such as task specialization, data efficiency, quicker training times, model customization, and resource efficiency. In some embodiments, fine-tuning may be particularly advantageous for niche applications and ongoing enhancement.

In other instances, the tools 220 can be utilized to pre-train and/or fine-tune the LLMs. The tools 220, or any subset thereof, may be standalone or part of a machine-learning operationalization framework, inclusive of hardware components like processors (e.g., CPU, GPU, TPU, FPGA, or any combination), memory, and storage. This framework operates software or computer program instructions (e.g., TensorFlow, PyTorch, Keras, etc.) to execute arithmetic, logic, input/output commands for training, validating, and deploying machine-learning models in a production environment. In certain instances, the tools 220 implement the training, validating, and deploying of the models using a cloud platform such as Oracle Cloud Infrastructure (OCI). Leveraging a cloud platform can make machine-learning more accessible, flexible, and cost-effective, which can facilitate faster model development and deployment for developers.

The tools 220 further include a prompt-based agent composition unit for creating agents and their associated actions that an end-user can end up invoking. An agent is a container of agent actions and can be part of one or more digital assistants. Each digital assistant may contain one or more agents through a digital assistant relation, which is the intersection entity that links an agent to a digital assistant. The agent and digital assistant are implemented as bot subtypes and may be persisted into an existing BOTS table. This has advantages in terms of reuse of design-time code (e.g., Java code) and UI artefacts.

An agent action is of a specific action type (e.g., knowledge, service or API, LLM, etc.) and contains a description and schema (e.g., JSON schema) which defines the action parameters. The action description and parameters schema are indexed by semantic index and sent to the planner to select the appropriate action(s) to execute. The action parameters are key-value pairs that are input for the action execution. They are derived from the properties in the schema but may also include additional UI/dialog properties that are used for slot filling dialogs. The actions can be part of one or more classes. For example, some actions may be part of an application event subscription class, which defines an agent action that should be executed when an application event is received. The application event can be received in the form of un update application context command message. An application event property mapping class (part of the application event subscription class) specifically maps the application event payload properties to corresponding agent action parameters. An action can optionally be part of an action group. An action group may be used when importing a plugin manifest, or when importing an external API spec such as an Open API spec. An action group is particularly useful when re-importing a plugin or open API spec, so new actions can be added, existing actions can be updated, or actions that are no longer present in the new manifest or Open API spec can be removed. At runtime, an action group may only be used to limit the application context groups that are sent to the LLM as conversation context by looking up the action group name which corresponds to a context group context.

The agents (e.g., 401k Change Contribution Agent) may be primarily defined as a compilation of agent artifacts using natural language within the prompt-based agent composition unit. Users 210 can create functional agents quickly by providing agent artifact information, parameters, and configurations and by pointing to assets. The assets can be or include resources, such as APIs for interfacing with applications, files and/or documents for retrieving knowledge, data stores for interacting with data, and the like, available to the agents for the execution of actions. The assets are imported, and then the users 210 can use natural language again to provide additional API customizations for dialog and routing/reasoning. Most of what an agent does may involve executing actions. An action can be an explicit action that's authored using natural language (similar to creating agent artifacts—e.g., ‘What is the impact of XYZ on my 401k Contribution limit?’ action in the below ‘401k Contribution Agent’ figure) or an implicit action that is created when an asset is imported (automatically imported upon pointing to a given asset based on metadata and/or specifications associated with the asset—e.g., actions created for Change Contribution and Get Contribution API in the below ‘401k Contribution Agent’ figure). The design time user can easily create explicit actions. For example, the user can choose the ‘Rich Text’ action type (see Table 1 for a list of exemplary action types) and creates the name artifact ‘What is the impact of XYZ on my 401k Contribution limit?’ when the user learns that a new FAQ needs to be added, as it's not currently in the knowledge documents (assets) the agent references (thus was not implicitly added as an action).

TABLE 1

| | Action Type | Description |
|---|---|---|
| 1 | Prompt | The action is implemented using a prompt to an LLM. |
| 2 | Rich Text | The action is implemented using rich text. The |
| | | most common use case is FAQs. |
| 3 | Flow | The action is implemented using Visual Flow |
| | | Designer flow. May be used for complex cases where |
| | | the developer is not able to use the out-of-the-box |
| | | dialogue and dialog customizations. |

There are various ways in which the agents and assets can be associated or added to a digital assistant 215. In some instances, the agents can be developed by an enterprise and then added to a digital assistant using DABP 205. In other instances, the agents can be developed and created using DABP 205 and then added to a digital assistant created using DABP 205. In yet other instances, DABP 205 provides an online digital store (referred to as an “agent store”) that offers various pre-created agents directed to a wide range of tasks and actions. The agents offered through the agent store may also expose various cloud services. In order to add the agents to a digital assistant being generated using DABP 205, a user 210 of DABP 205 can access assets via tools 220, select specific assets for an agent, initiate a few mock chat conversations with the agent, and indicate that the agent is to be added to the digital assistant created using DABP 205.

Once deployed in a production environment, such as the architecture described with respect to FIG. 2, a digital assistant, such as digital assistant 215A built using DABP 205, can be used to perform various tasks via natural language-based conversations between the digital assistant 215A and its users 225. As described above, the digital assistant 215A illustrated in FIG. 2, can be made available or accessible to its users 225 through a variety of different channels, such as but not limited to, via certain applications, via social media platforms, via various messaging services and applications, and other applications or channels. A single digital assistant can have several channels configured for it so that it can be run on and be accessed by different services simultaneously.

As part of a conversation, a user 225 may provide one or more user inputs 230 to digital assistant 215A and get responses 235 back from digital assistant 215A via a user interface element such as a chat window. A conversation can include one or more of user inputs 230 and responses 235. Via these conversations, a user 225 can request one or more tasks to be performed by the digital assistant 215A and, in response, the digital assistant 215A is configured to perform the user-requested tasks and respond with appropriate responses to the user 225 using one or more LLMs 240. Conversations shown in the chat window can be organized by thread. For example, in some applications, a conversation related to one page of an application should not be mixed with a conversation related to another page of the application. The application and/or the plugins for the application define the thread boundaries (e.g., a set of (nested) plugins can run within their own thread). Effectively, the chat window will only show the history of messages that belong to the same thread. Setting and changing the thread can be performed via the application and/or the plugins using an update application context command message. Additionally or alternatively, the thread can be changed via an execution plan orchestrator when a user query is matched to a plugin semantic action and the plugin runs in a thread different than the current thread. In this case, the planner changes threads, so that any messages sent in response to the action being executed are shown in the correct new thread. Per agent dialog thread, the following information can be maintained by the digital assistant: the application context, the LLM conversation history, the conversation history with the user, and the agent execution context which holds information about the (stacked) execution plan(s) related to this thread.

User inputs 230 are generally in a natural language form and are referred to as utterances, which may also be referred to as prompts, queries, requests, and the like. The user inputs 230 can be in text form, such as when a user types in a sentence, a question, a text fragment, or even a single word and provides it as input to digital assistant 215A. In some embodiments, a user input 230 can be in audio input or speech form, such as when a user says or speaks something that is provided as input to digital assistant 215A. The user inputs 230 are typically in a language spoken by the user 225. For example, the user inputs 230 may be in English, or some other language. When a user input 230 is in speech form, the speech input is converted to text form user input 230 in that particular language and the text utterances are then processed by digital assistant 215A. Various speech-to-text processing techniques may be used to convert a speech or audio input to a text utterance, which is then processed by digital assistant 215A. In some embodiments, the speech-to-text conversion may be done by digital assistant 215A itself. For purposes of this disclosure, it is assumed that the user inputs 230 are text utterances that have been provided directly by a user 225 of digital assistant 215A or are the results of conversion of input speech utterances to text form. This however is not intended to be limiting or restrictive in any manner.

The user inputs 230 can be used by the digital assistant 215A to determine a list of candidate agents 245A-N. The list of candidate agents (e.g., 245A-N) includes agents configured to perform one or more actions that could potentially facilitate a response 235 to the user input 230. The list may be determined by running a search, such as a semantic search, on a context and memory store that has one or more indices comprising metadata for all agents 245 available to the digital assistant 215A. Metadata for the candidate agents 245A-N in the list of candidate agents is then combined with the user input to construct an input prompt for the one or more LLMs 240.

Digital assistant 215A is configured to use one or more generative models such as LLMs 240 to apply NLP techniques to text and/or speech to understand the input prompt and apply natural language understanding (NLU) including syntactic and semantic analysis of the text and/or speech to determine the meaning of the user inputs 230. Determining the meaning of the utterance may involve identifying the goal of the user, one or more intents of the user, the context surrounding various words or phrases or sentences, one or more entities corresponding to the utterance, and the like. The NLU processing can include parsing the received user inputs 230 to understand the structure and meaning of the utterance, refining and reforming the utterance to develop a better understandable form (e.g., logical form) or structure for the utterance. The NLU processing performed can include various NLP-related processing such as sentence parsing (e.g., tokenizing, lemmatizing, identifying part-of-speech tags for the sentence, identifying named entities in the sentence, generating dependency trees to represent the sentence structure, splitting a sentence into clauses, analyzing individual clauses, resolving anaphoras, performing chunking, and the like). In certain instances, the NLU processing, or any portions thereof, is performed by the LLMs 240 themselves. In other instances, the LLMs 240 use other resources to perform portions of the NLU processing. For example, the syntax and structure of an input utterance sentence may be identified by processing the sentence using a parser, a part-of-speech tagger, a named entity recognition model, a pretrained language model such as BERT, or the like.

Upon understanding the meaning of an utterance, the one or more LLMs 240 generate an execution plan that identifies one or more agents (e.g., agent 245A) from the list of candidate agents to execute and perform one or more actions or operations responsive to the understood meaning or goal of the user. The one or more actions or operations are then executed by the digital assistant 215A on one or more assets (e.g., asset 250A-knowledge, API, SQL operations, etc.) and/or the context and memory store. The execution of the one or more actions or operations generates output data from one or more assets and/or relevant context and memory information from a context and memory store comprising context for a present conversation with the digital assistant 215A. The output data and relevant context and memory information are then combined with the user input 230 to construct an output prompt for one or more LLMs 240. The LLMs 240 synthesize the response 235 to the user input 230 based on the output data and relevant context and memory information, and the user input 230. The response 235 is then sent to the user 225 as an individual response or as part of a conversation with the user 225.

For example, a user input 230 may request a pizza to be ordered by providing an utterance such as “I want to order a pizza.” Upon receiving such an utterance, digital assistant 215A is configured to understand the meaning or goal of the utterance and take appropriate actions. The appropriate actions may involve, for example, providing responses 235 to the user with questions requesting user input on the type of pizza the user desires to order, the size of the pizza, any toppings for the pizza, and the like. The questions requesting user may be generated by executing an action via an agent (e.g., agent 245A) on a knowledge asset (e.g., a menu for a pizza restaurant) to retrieve information that is pertinent to ordering a pizza (e.g., to order a pizza a user must provide type, seize, topping, etc.). The responses 235 provided by digital assistant 215A may also be in natural language form and typically in the same language as the user input 230. As part of generating these responses 235, digital assistant 215A may perform natural language generation (NLG) using the one or more LLMs 240. For the user ordering a pizza, via the conversation between the user and digital assistant 215A, the digital assistant 215A may guide the user to provide all the requisite information for the pizza order, and then at the end of the conversation cause the pizza to be ordered. The ordering may be performed by executing an action via an agent (e.g., agent 245A) on an API asset (e.g., an API for ordering pizza) to upload or provide the pizza order to the ordering system of the restaurant. Digital assistant 215A may end the conversation by generating a final response 235 providing information to the user 225 indicating that the pizza has been ordered.

While the various examples provided in this disclosure describe and/or illustrate utterances in the English language, this is meant only as an example. In certain embodiments, digital assistants 215 are also capable of handling utterances in languages other than English. Digital assistants 215 may provide subsystems (e.g., components implementing NLU functionality) that are configured for performing processing for different languages. These subsystems may be implemented as pluggable units that can be called using service calls from an NLU core server. This makes the NLU processing flexible and extensible for each language, including allowing different orders of processing. A language pack may be provided for individual languages, where a language pack can register a list of subsystems that can be served from the NLU core server.

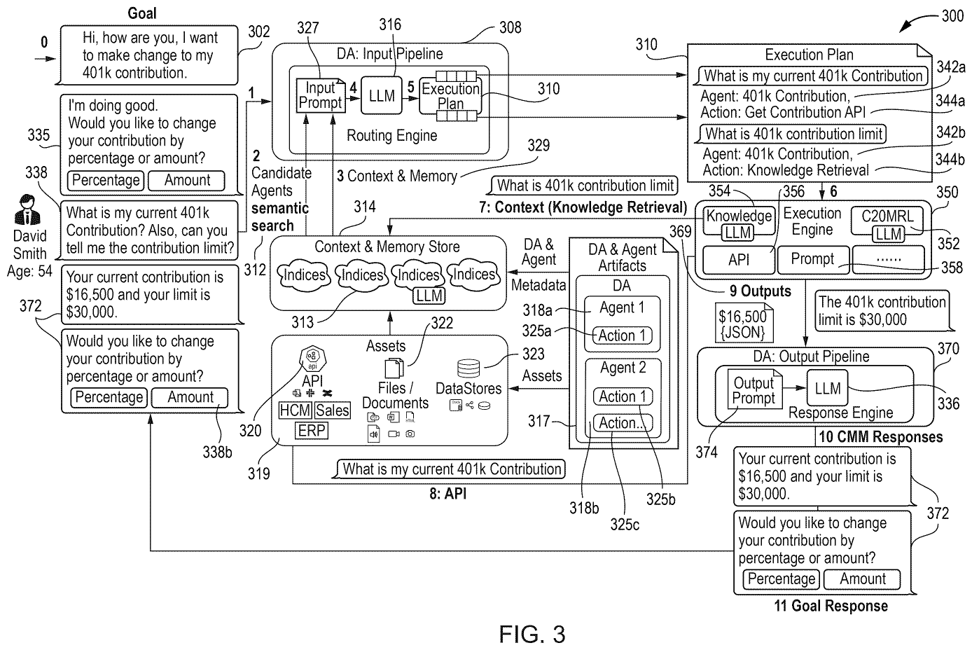

While the embodiment in FIG. 2 illustrates the digital assistant 215A including one or more LLMs 240 and one or more agents 245A-N, this is not intended to be limiting. A digital assistant can include various other components (e.g., other systems, subsystems, and generative models as described in greater detail with respect to FIG. 3) that provide the functionalities of the digital assistant. The digital assistant 215A and its systems and subsystems may be implemented only in software (e.g., code, instructions stored on a computer-readable medium and executable by one or more processors), in hardware only, or in implementations that use a combination of software and hardware.

FIG. 3 is an example of an architecture for a computing environment 300 for a digital assistant implemented with generative artificial intelligence in accordance with various embodiments. As illustrated in FIG. 3, an infrastructure and various services and features can be used to enable a user to interact with a digital assistant (e.g., digital assistant 215A described with respect to FIG. 2) based at least in part on a series of prompts such as a conversation and/or a series of actions such as interactions with a user interface. The following is a detailed walkthrough of a conversation flow and the role and responsibility of the components, services, models, and the like of the computing environment 300 within a conversation flow. In this walkthrough, it is assumed that a user “David” is interested in making a change to his 401k contribution, and in an utterance 302, David provides the following input: Hi, how are you, I want to make a change to my 401k contribution. The utterance 302 can be communicated to the digital assistant (e.g., via a digital assistant user interface such as a text dialogue box or microphone). At this stage upon receipt of the utterance 302, a sessionizer creates a new session or retrieves the current session context and a user message publisher updates session transcript and LLM message history with the new user message (e.g., utterance 302).

In instances where the user provides the utterance 302 and/or performs an action while using an application supported by a digital assistant, the application issues update application context commands as the user interacts with the application (e.g., provides an utterance via text or audio, triggers a user interface element, navigates between pages of the application, and the like). Whenever an update application context command message is received by the digital assistant from the application, the application context processor (part of the context manager) is implemented. The application context processor performs the following tasks: (i) manages dialog threads based on the application context message, e.g., if the threadId specified with the message doesn't exist yet, a new dialog thread is created and made current, and if the threadId already exists, the corresponding dialog thread is made current, (ii) creates or updates the application context object for the current dialog thread, (iii) if a service call ID such as a REST request ID is included, the application context may be enriched (as described in greater detail herein). As should be understood, the application context only contains information that reflects the state of the application user interface and plugins (if available), it does not contain other state information (e.g., user or page state information/context).

Is some instances, when an update application context command message is received, an application event processor checks on whether the update application context command message includes an event definition. The event is uniquely identified by the following properties in the message payload: (i) context: the context path and/or the plugin path (For a top-level workspace plugin the context is set to the plugin name, for nested plugins the plugin path is included where plugins are separated with a slash, for example Patient/Vitalschart), (ii) eventType: the type of event can be one of the built in events or a custom event, and (iii) semantic object: the semantic object to which the event applies. An event can be mapped to one or more actions, and the message payload properties can be mapped to action parameters. This mapping takes place through an application event subscription. Each property in the message payload can be mapped to an agent action parameter using an application event property mapping.

In some instances, the utterance 302 and/or action performed by the user is provided directly as input to a routing engine 308 (also referred to as a planner). In other instances where the application event processor is implemented, the utterance 302 and/or action performed by the user is provided as input to the routing engine 308 when the application event processor determines an event such as receipt of utterance 302 is mapped to an agent or action associated with the digital assistant. The routing engine 308 is used by the digital assistant to create an execution plan 310 with specified parameters either from the utterance 302, the action performed by the user, the context, or any combination thereof. The execution plan 310 identifies one or more agents and/or one or more actions for the one or more agents to execute in response to the utterance 302 and/or action performed by the user.

A two-step approach can be taken via the routing engine 308 to generate the execution plan 310. First, a search 312 can be performed to identify a list of candidate agents and/or actions. The search 312 comprises running a query on indices 313 (e.g., semantic indices) of a context and memory store 314 based on the utterance 302 and/or action performed by the user. In some instances, the search 312 is a semantic search performed using words from the utterance 302 and/or representative of the action performed by the user. The semantic search uses NLP and optionally machine learning techniques to understand the meaning of the utterance 302 and/or action performed by the user and retrieve relevant information from the context and memory store 314. In contrast to traditional keyword-based searches, which rely on exact matches between the words in the query and the data in the context and memory store 314, a semantic search takes into account the relationships between words, the context of the query and/or action, synonyms, and other linguistic nuances. This allows the digital assistant to provide more accurate and contextually relevant results, making it more effective in understanding the user's intent in the utterance 302 and/or action performed by the user.

In order to run the query, the routing engine 308 calls the context and memory store 314 (e.g., a semantic index of the context and memory store 314) to get the list of candidate agents and/or actions. The following information is passed in the call: (i) the ID of the digital assistant (the ID scopes the set of agent and/or actions the semantic index will search for and thus the agents and/or actions must be part of the digital assistant), and (ii) the last X number of user messages and/or actions (e.g., X can be set to the last 5 turns), which can be configurable through the digital assistant settings. Upon receiving the list of candidate agents and/or actions, the routing engine 308 can identify an associated schema with the actions and perform slot-filling to determine any missing input arguments for the schema.

The context and memory store 314 is implemented using a data framework for connecting external data to one or more generative models such as LLMs 316 to make it easy for users to plug in custom data sources. The data framework provides rich and efficient retrieval mechanisms over data from various sources such as files, documents, datastores, APIs, and the like. The data can be external (e.g., enterprise assets) and/or internal (e.g., user preferences, memory, digital assistant, and agent metadata, etc.). In some instances, the data comprises metadata extracted from artifacts 317 associated with the digital assistant and its agents 318 (e.g., 318a and 318b). The artifacts 317 for the digital assistant include information on the general capabilities of the digital assistant and specific information concerning the capabilities of each of the agents 318 (e.g., actions) available to the digital assistant (e.g., agent artifacts). Additionally or alternatively, the artifacts 317 can encompass parameters or information associated with the artifacts 317 and that can be used to define the agents 318 in which the parameters or information associated with the artifacts 317 can include a name, a description, one or more actions, one or more assets, one or more customizations, etc. In some instances, the data further includes metadata extracted from assets 319 associated with the digital assistant and its agents 318 (e.g., 318a and 318b). The assets 319 may be resources, such as APIs 320, files and/or documents 322, data stores 323, and the like, available to the agents 318 for the execution of actions (e.g., actions 325a, 325b, and 325c). The data is indexed in the context and memory store 314 as indices 313, which are data structures that provide a fast and efficient way to look up and retrieve specific data records within the data. Consequently, the context and memory store 314 provides a searchable comprehensive record of the capabilities of all agents and associated assets that are available to the digital assistant for responding to the request and/or action.

The response of context and memory store 314 is converted into a list of agent and/or action instances that are not just available to the digital assistant for responding to the request but also potentially capable of facilitating the generation of a response to the utterance 302 and/or action performed by the user. The list of candidate agents and/or actions includes the metadata (e.g., metadata extracted from artifacts 317 and assets 319) from the context and memory store 314 that is associated with each of the candidate agents and/or actions. The list can be limited to a predetermined number of candidate agents and/or actions (e.g., top 10) that satisfy the query or can include all agents and/or actions that satisfy the query. The list of candidate agents and/or actions with associated metadata is appended to the utterance 302 and/or action performed by the user to construct an input prompt 327 for the LLM 316. The search 312 is important to the digital assistant because it filters out agents and/or actions that are unlikely to be capable of facilitating the generation of a response to the utterance 302 and/or action performed by the user. This filter ensures that the number of tokens (e.g., word tokens) generated from the input prompt 327 remains under a maximum token limit or context limit set for the LLM 316. Token limits represent the maximum amount of text that can be inputted into an LLM. This limit is of a technical nature and arises due to computational constraints, such as memory and processing resources, and thus makes certain that the LLMs can take the input prompt as input.

In some instances, one or more knowledge actions are additionally appended to the list of candidate agents and the utterance 302. The knowledge actions allow for additional knowledge to be acquired that is pertinent to the utterance 302 and/or action performed by the user (this knowledge is typically outside the scope of the knowledge used to train an LLM of the digital assistant). The are two types of knowledge action sources: (i) structure: the knowledge source defines a list of pre-defined questions that the user might ask and exposes them as some APIs (e.g., Multum), and (ii) unstructured: with the knowledge source, the user has unlimited ways to ask questions and the knowledge source exposes a generic query interface (e.g., medical documents (SOAP notes, discharge summary, etc.)).

In some instances, conversation context 329 concerning the utterance 302 are additionally appended to the list of candidate agents and the utterance 302. The conversation context 329 can be retrievable from one or more sources including the context and memory store 314, and includes user session information, dialog state, conversation or contextual history, application context, page context, user information, or any combination thereof. For example, the conversation context 329 can include: the current date and time, needed to resolve temporal references in user query like “yesterday”, or “next Thursday”, additional context, which contains information such as user profile properties and application context groups with semantic object properties, and the chat history with the digital assistant (and/or other digital assistant or system internal or external to the computing environment 300.

The second step of the two-step approach is for the LLM 316 to generate an execution plan 310 based on the input prompt 327. The LLM 316 can be invoked by creating an LLM chat message with role system passing in the input prompt 327, converting the candidate agents and/or actions into LLM function definitions, retrieving a proper LLM client based on the LLM configuration options, optionally transforming the input prompt 327, LLM chat message, etc. into a proper format for the LLM client, and sending the LLM chat message to the LLM client for invoking the LLM 316. The LLM client then sends back an LLM success response in CLMI format or a provider specific response is converted back to the LLM success response in CLMI format using an adapter such as OpenAIAdapter (or send back or is converted to an LLM error response in case an unexpected error occurred). An LLM call instance is created and added to the conversation history which captures all the request and response details including the execution time.

The LLM 316 has a deep generative model architecture (e.g., a reversible or autoregressive architecture with) for generating the execution plan 310. In some instances, the LLM 316 has over 100 billion parameters and generates the execution plan 310 using autoregressive language modeling within a transformer architecture, allowing the LLM 316 to capture complex patterns and dependencies in the input prompt 327. The LLM's 316 ability to generate the execution plan 310 is a result of its training on diverse and extensive textual data, enabling the LLM to understand human language across a wide range of contexts. During training, the LLM 316 learns to predict the next word in a sequence given the context of the preceding words. This process involves adjusting the model's parameters (weights and biases) based on the errors between its predictions and the actual next words in the training data. When the LLM 316 receives an input such as the input prompt 327, the LLM 316 tokenizes the text into smaller units such as words or sub-words. Each token is then represented as a vector in a high-dimensional space. The LLM 316 processes the input sequence token by token, maintaining an internal representation of context. The LLM's 316 attention mechanism allows it to weigh the importance of different tokens in the context of generating the next word. For each token in the vocabulary, the LLM 316 calculates a probability distribution based on its learned parameters. This probability distribution represents the likelihood of each token being the next word given the context. For example, to generate the execution plan 310, the LLM 316 samples a token from the calculated probability distribution. The sampled token becomes the next word in the generated sequence. This process is repeated iteratively, with each newly generated token influencing the context for generating the subsequent token. The LLM 316 can continue generating tokens until a predefined length or stopping condition is reached.

In some instances, as illustrated in FIG. 3, the LLM 316 may not be able to generate a complete execution plan 310 because it is missing information such as if more information is required to determine an appropriate agent for the response, execute one or more actions, or the like. In this particular instance, the LLM 316 has determine that in order to change the 401k contribution as request by the user, it is necessary to understand whether the user would like to change the contribution by a percentage or certain currency amount. In order to obtain this information, the LLM 316 (or another LLM such as LLM 336) generates end-user response 335 (I'm doing good. Would you like to change your contribution by percentage or amount? [Percentage] [Amount]) to the input prompt 327 that can obtain the missing information such that the LLM 316 is able to generate a complete execution plan 310. In some instances, the response may be rendered within a dialogue box of a UI having one or more UI elements allowing for an easier response by the user. In other instances, the response may be rendered within a dialogue box of a UI allowing for the user to reply using the dialogue box (or alternative means such as a microphone). In this particular instance, the user responds with an additional query 338 (What is my current 401k Contribution? Also, can you tell me the contribution limit?) to gather additional information such that the user can reply to the response 335. The subsequent response-additional query 338—is input into the routing engine 308 and the same processes described above with respect to utterance 302 are executed but this time with the context of the prior utterances/replies (e.g., utterance 302 and response 335) from the user's conversation with the digital assistant. This time, as illustrated in FIG. 3, the LLM 316 is able to generate a complete execution plan 310 because it has all the information it needs.

In some instances, the utterance 302 by the user may be determined by the LLM 316 to be non-sequitur (i.e., an utterance that does not logically follow from the previous utterance in a dialogue or conversation). In such an instance, an execution plan orchestrator can be used to handle the switch among different dialog paths. The execution plan orchestrator is configured to track all the ongoing conversation paths, create a new entry if a new dialog path is created and pause the current ongoing conversation if any, remove the entry if the conversation completes based on the metadata of the new action or user preference, it might generate a prompt message when starting a non-sequitur or resuming the previous one, manage the dialog for the prompt message and either proceed or restore the current conversation, confirm or cancel when the user responds to the prompt for the non-sequitur, and manages a cancel or exit from a dialog.

The execution plan 310 includes an ordered list of agents and/or actions that can be used and/or executed to sufficiently respond to the request such as the additional query 338. For example, and as illustrated in FIG. 3, the execution plan 310 can be an ordered list that includes a first agent 342a capable of executing a first action 344a via an associated asset and a second agent 342b capable of executing a second action 344b via an associated asset. The agents, and by extension the actions, may be ordered to cause the first action 344a to be executed by the first agent 342a prior to causing the second action 344b to be executed by the second agent 342b. In some instances, the execution plan 310 may be ordered based on dependencies indicated by the agents and/or actions included in the execution plan 310. For example, if executing the second agent 342b is dependent on, or otherwise requires, an output generated by the first agent 342a executing the first action 344a, then the execution plan 310 may order the first agent 342a and the second agent 342b to comply with the dependency. As should be understood, other examples of dependencies are possible.

The execution plan 310 is then transmitted to an execution engine 350 for implementation. The execution engine 350 includes a number of engines, including a natural language-to-programming language translator 352, a knowledge engine 354, an API engine 356, a prompt engine 358, and the like, for executing the actions of agents and implementing the execution plan 310. For example, the natural language-to-programming language translator 352, such as a NL2SQL model, may be used by an agent to translate natural language into a system programming language (e.g., SQL) and execute the system programming language (e.g., execute an SQL query) on an asset 319 such as data stores 323 to execute actions and/or obtain data or information. The knowledge engine 354 may be used by an agent to obtain data or information from the context and memory store 314 or an asset 319 such as files/documents 322. The API engine 356 may be used by an agent to call an API 320 and interface with an application such as retirement fund account management application to execute actions and/or obtain data or information. The prompt engine 358 may be used by an agent to construct a prompt for input into an LLM such as an LLM in the context and memory store 314 or an asset 319 to execute actions and/or obtain data or information.

The execution engine 350 implements the execution plan 310 by running each agent and executing each action in order based on the ordered list of agents and/or actions using the appropriate engine(s). To facilitate this implementation, the execution engine 350 is communicatively connected (e.g., via a public and/or provue network) with the agents (e.g., 342a, 342b, etc.), the context and memory store 314, and the assets 319. For example, as illustrated in FIG. 3, when the execution engine 350 implements the execution plan 310, it will first execute the agent 342a and action 344a using API engine 356 to call the API 320 and interface with a retirement fund account management application to retrieve the user's current 401k contribution. Subsequently, the execution engine 350 can execute the agent 342b and action 344b using knowledge engine 354 to retrieve knowledge on 401k contribution limits. In some instances, the knowledge is retrieved by knowledge engine 354 from the assets 319 (e.g., files/documents 322). In other instances (as in this particular instance), the knowledge is retrieved by knowledge engine 354 from the context and memory store 314. Knowledge retrieval and action execution using the context and memory store 314 may be implemented using various techniques including internal task mapping and/or machine learning models such as additional LLM models. For example, the query and associated agent for “What is 401k contribution limit” may be mapped to a ‘semantic search’ knowledge task type for searching the indices 313 within the context and memory store 314 for a response to a given query. By way of another example, a request such as “Can you summarize the key points relating to 401k contribution” can be or include a ‘summary’ knowledge task type that may be mapped to a different index within the context and memory store 314 having an LLM trained to create a natural language response (e.g., summary of key points relating to 401k contribution) to a given query. Over time, a library of generic end-user task or action types (e.g., semantic search, summarization, compare/contrast, heterogeneous data synthesis, etc.) may be built to ensure that the indices and models within the context and memory store 314 are optimized to the various task or action types.

The result of implementing the execution plan 310 is output data 369 (e.g., results of actions, data, information, etc.), which is transmitted to an output pipeline 370 for generating end-user responses 372. For example, the output data 369 from the assets 319 (knowledge, API, dialog history, etc.) and relevant information from the context and memory store 314 can be transmitted to the output pipeline 370. The output data 369 is appended to the utterance 302 to construct an output prompt 374 for input to the LLM 336. In some instances, context 329 concerning the utterance 302 are additionally appended to the output data 369 and the utterance 302. The context 329 is retrievable from the context and memory store 314 and includes user session information, dialog state, conversation or contextual history, user information, or any combination thereof. The LLM 336 generates responses 372 based on the output prompt 374. In some instances, the LLM 336 is the same or similar model as LLM 316. In other instances, the LLM 336 different from LLM 316 (e.g., trained on a different set of data, a different architecture, trained for a one or more different tasks, etc.). In either instance, the LLM 336 has a deep generative model architecture (e.g., a reversible or autoregressive architecture with) for generating the responses 372 using similar training and generative processes described above with respect to LLM 316. In some instances, the LLM 336 has over 100 billion parameters and generates the responses 372 using autoregressive language modeling within a transformer architecture, allowing the LLM 336 to capture complex patterns and dependencies in the output prompt 374.

In some instances, the end-user responses 372 may be in the format of a Conversation Message Model (CMM) and output as rich multi-modal responses. The CMM defines the various message types that the digital assistant can send to the user (outbound), and the user can send to the digital assistant (inbound). In certain instances, the CMM identifies the following message types:
text: Basic text message
card: A card representation that contains a title and, optionally, a description, image, and link
attachment: A message with a media URL (file, image, video, or audio)
location: A message with geo-location coordinates
postback: A message with a postback payload
   Messages that are defined in CMM are channel-agnostic and can be created using CMM syntax. The channel-specific connectors transform the CMM message into the format required by the specific channel, allowing a user to run the digital assistant on multiple channels without the need to create separate message formats for each channel.

Lastly, the output pipeline 370 transmits the responses 372 to the end user such as via a user device or interface. In some instances, the responses 372 are rendered within a dialogue box of a GUI allowing for the user to view and reply using the dialogue box (or alternative means such as a microphone). In other instances, the responses 372 are rendered within a dialogue box of a GUI having one or more GUI elements allowing for an easier response by the user. In this particular instance, a first response 372 (What is my current 401k Contribution? Also, can you tell me the contribution limit?) to the additional query 338 is rendered within the dialogue box of a GUI. Additionally, in order to follow-up on obtaining information still required for the initial utterance 302, the LLM 336 generates another response 372 prompting the user for the missing information (Would you like to change your contribution by percentage or amount? [Percentage] [Amount]).

While the embodiment of computing environment 300 in FIG. 3 illustrates the digital assistant interacting in a particular conversation flow, this is not intended to be limiting and is merely provided to facilitate a better understanding of the role and responsibility of the components, services, models, and the like of the computing environment 300 within the conversation flow.

### Entity Linking Pipeline and Processes

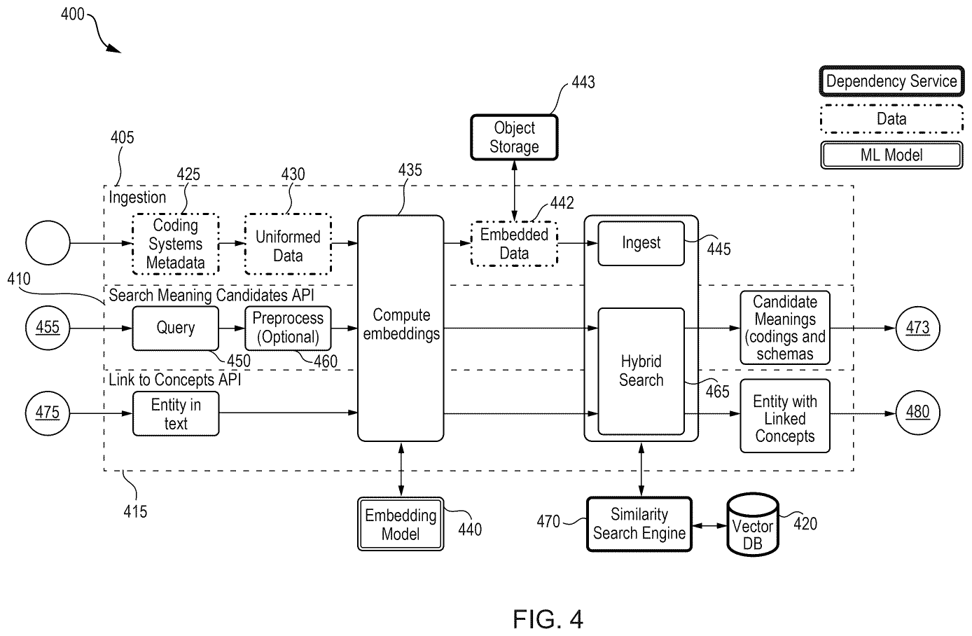

FIG. 4 is an example of an architecture for a computing environment 400 for a Clinical Linking Service (CLS) that leverages an advanced, embedding-based similarity search architecture to deliver a highly accurate, scalable, and flexible solution for normalizing clinical text, in accordance with various embodiments. Computing environment 400 may be implemented as a system comprising software, hardware, firmware, or any combination thereof. In certain embodiments, the system is realized as a set of software modules executing on one or more processors, such as in a server, personal computer, or distributed computing environment. Alternatively or additionally, the system can be implemented as part of a cloud-based infrastructure (e.g., Infrastructure as a Service (IaaS), for example, see description of FIGS. 7-11), where scalable computing resources and storage capabilities are leveraged to process and manage high volumes of user requests and database queries. This architecture supports high availability, rapid response times, and robust security enabling the framework to deliver real-time or near-real-time interactions for a diverse set of users. The system may be embodied in, or accessible via, various physical devices and platforms, including but not limited to computers, mobile devices, appliances, networked embedded systems, or vehicles. Deployment can occur on-premises, in public or private clouds, or in a hybrid configuration.

Computing environment 400 comprises three pipelines: (i) ingestion pipeline 405, (ii) search meaning candidates API pipeline 410, and (iii) link to concepts API pipeline 415. The ingestion pipeline 405 is executed in order to provide the ingest coding information into the vector database 420. The search meaning candidates API pipeline 410 is executed as a microservice utilizing an API call to take a broad user query and return a ranked list of potential matching concepts (both codes and schemas). The search meaning candidates API pipeline 410 is optimized for recall, ensuring that all potentially relevant meanings are captured for further processing by other services, such as a schema synthesizer or NL2SQL services. The link to concepts API pipeline 415 is more targeted. The link to concepts API pipeline 415 takes specific, pre-identified entities from a text and links them to the most relevant codes (e.g., based on semantic proximity) within specified coding systems (e.g., SNOMED CT, LOINC, ICD-10-CM). This function is focused on achieving a balance of high precision and recall.

The ingestion pipeline 405 is a preparatory, offline process responsible for populating the vector database 420. The pipeline begins at a data source interface 425, which is configured to connect to and retrieve data (e.g., concept metadata, hierarchical relationships, and cross-mappings) from a plurality of standardized medical coding systems, such as SNOMED CT, ICD-10-CM, LOINC, and RxNorm. Data source interface 425 may obtain this information by accessing publicly available data files (e.g., CSV, XML, JSON) distributed by the respective organizations, or by invoking dedicated APIs or web services to download and synchronize the latest releases and updates. The interface is capable of parsing not only the core concepts and their unique identifiers but also extracting system-specific fields such as hierarchical relationships (e.g., “is-a” and attribute relationships in SNOMED CT), synonym lists, preferred terms, and mappings to other coding systems.

The retrieved, system-specific data is then provided to a uniformed data processing module 430. This module is configured to normalize and unify the heterogeneous data across all coding systems into a consistent, structured format. The normalization process may involve extracting and standardizing fields such as concept identifiers, official names, display texts, synonyms, definitions, relationships, and cross-references. The module harmonizes field names and values, resolves redundancies where identical clinical concepts exist across multiple systems, and creates a unified schema that abstracts away system-specific peculiarities. For example, for the concept of myocardial infarction, the logical definition in SNOMED CT is an ischemic heart disease (disorder) and a myocardial necrosis (disorder), with associated attributes such as finding site (myocardium structure) and morphology (infarct), all mapped to standardized codes (e.g., SNOMED CT code 22298006). The unified data processing module 430 consolidates this metadata and structure, ensuring interoperability and consistency across concepts from disparate coding systems.

The unified and normalized data produced by the uniformed data processing module 430 is next supplied to an embedding generation module 435. Embedding generation module 435 is configured to transform each concept's structured data (including official names, synonyms, definitions, and relational attributes) into a numerical representation suitable for similarity-based computation. This transformation is accomplished by invoking a pre-trained semantic embedding model 440, such as SapBert, which has been trained on large biomedical corpora and is capable of encoding complex semantic relationships inherent in medical terminology. The embedding model 440 accepts the textual and relational data for each concept as input and, through a series of neural network computations, produces a corresponding high-dimensional vector (often comprising hundreds of dimensions). This vector captures not only the literal meaning of the concept but also its contextual, hierarchical, and relational properties, allowing for sophisticated semantic comparisons.

Once the vector representation for each concept is generated, the concept and its embedding (embedding data 442) are stored in object storage 443 and forwarded to an indexing module 445. More specifically, the concept including its contextual, hierarchical, and relational properties may be stored as part of a semantic object schema in the object storage 443. In runtime, this semantic object schema will load into memory. The schema will have information from semantic object level to attribute level to public coding system or proprietary code set level information and is tenant agnostic. Indexing module 445 is responsible for organizing and persisting these vectors within the vector database 420, which may be implemented using a specialized vector search engine such as Qdrant or OpenSearch. The indexing process involves associating each concept's unique identifier, metadata, and semantic vector with an entry in the database, structuring the data so that it can be efficiently retrieved during similarity search operations. Indexing module 445 may also perform optimization steps such as normalization of vectors, storage of auxiliary metadata for filtering and faceted search, and creation of secondary indexes to support rapid lookup of concept attributes.

The vector database 420 thus becomes a comprehensive repository of standardized medical concepts, each represented by a semantically rich vector and associated metadata (e.g., a pre-indexed vector database comprising vector representations for medical codings and semantic object schemas). This architecture enables real-time, high-performance similarity searches, where incoming queries or entity vectors can be rapidly compared against the indexed vectors to identify the most relevant concepts based on semantic proximity. The result is a scalable, flexible infrastructure that supports downstream API pipelines, such as search meaning candidates API pipeline 410 and link to concepts API pipeline 415, with robust semantic normalization and interoperability across diverse medical coding systems.

The search meaning candidates API pipeline 410 represents a real-time process initiated by a subsystem within computing environment 400. The process begins when a request, containing an unstructured text query 450 (for example, “patient has history of UTI”), is received at an API endpoint 455. Upon receipt, the query 450 may be routed to a preprocessing module 460, which is configured to execute a series of preparatory operations. These operations may be performed by a Knowledge Processing Engine (KPE) and may include key word or phrase segmentation to identify and isolate medically relevant terms and entities, syntactic parsing to structure the input, and data sanitization to remove or encrypt any Protected Health Information (PHI), thereby ensuring compliance with privacy standards. Preprocessing module 460 may also transform the raw query into a standardized schema that aligns with the requirements of downstream analytics or AI/ML models, facilitating consistent and reliable data handling.

The query 450 or resulting processed text is then supplied to the embedding generation module 435. This module utilizes a pre-trained semantic embedding model 440 such as SapBert capable of encoding the medical text into a high-dimensional vector representation. As discussed above, the model is trained on extensive biomedical literature and clinical datasets, enabling it to capture deep semantic relationships and context, including synonyms, abbreviations, hierarchical relationships, and latent structure within medical language. The embedding generation module 435 transforms the processed query or key phrases into a numerical vector that represents its semantic meaning in a format suitable for similarity-based computation.

This query vector, along with a search configuration provided in the API request (defining parameters such as target index, number of results, confidence thresholds, and algorithm weights), is forwarded to a hybrid search module 465. The hybrid search module 465 orchestrates a multi-modal search process using a similarity search engine 470, such as Qdrant or OpenSearch, which is specifically optimized for rapid, large-scale vector similarity operations. The hybrid search combines k-Nearest Neighbors (kNN) search, which identifies concepts whose embeddings are nearest in semantic space to the query vector, and keyword-based search, which matches concepts based on textual overlap and known synonyms. The hybrid search module 465 applies configurable weights to both search strategies, allowing for fine-tuned balancing between semantic and lexical matching.

The similarity search engine 470 executes these searches against the indexed vectors stored in the vector database 420, which contains vector representations for both granular medical codes and higher-level semantic object schemas, to efficiently retrieve a multifaceted set of candidate meanings 472. This retrieved set includes entities whose vector representations are most similar or relevant to the query vector and/or which match the query keywords. The hybrid search module 465 then processes and ranks these results, organizing them based on search type (e.g., kNN and keyword), computed similarity scores, and type (i.e., schema or coding), while optionally applying additional filters or thresholds defined in the search configuration.

The resulting output, returned as a structured response through the API endpoint 473 (same or different endpoint from that of endpoint 455), is not merely a flat list of concepts but may be intelligently partitioned into two primary components: a ranked list of candidate codings and a list of optional candidate schemas. The candidate schemas represent tenant-agnostic, semantic object structures, which may be loaded into memory at runtime from an object store. The candidate schemas define high-level clinical concepts, from the semantic object level down to the attribute level. These schemas provide a structural and contextual framework. The candidate codings, in turn, represent the specific, granular concepts from standardized terminologies (e.g., SNOMED CT, LOINC, ICD-10-CM) that correspond to the terms in the user's query. This dual-component response provides the calling subsystem not only with potential standardized codes but also with the structural context (the schema) in which those codes might fit. This output is specifically designed for consumption by downstream processes, such as a schema synthesizer, which can use the identified schemas and codings to construct a complete, structured, and machine-readable representation of the user's original query, thereby enabling more advanced applications like automated clinical decision support or generative AI-driven summarization and database querying as described with respect to FIGS. 1-3.

To further illustrate the operation of the search meaning candidates API pipeline 410, the following Table 2 provides a concrete, step-by-step example of the system processing a natural language query. As shown, the pipeline takes an unstructured query, performs optional preprocessing to segment the query into key clinical terms, computes vector embeddings for these terms, and then executes a search using a detailed search configuration. The final output is a rich, structured JSON object containing the ranked candidate meanings, which are intelligently separated into candidate codings and schemas, complete with identifiers, scores, and other relevant metadata. This example demonstrates the end-to-end data flow and the sophisticated, structured nature of the results provided by the API.

TABLE 2

| Search | Preprocess | | |
|---|---|---|---|
| Query | (Optional) | Search config | Candidate Meanings |
| has UTI in | Segments: | json { “indexName”: | json { “searchedMeanings”: { “schemas”: [ { }, { } ], |
| she the | “UTI”, | “meaning_index”, | “codings”: [ { “nl”: “uti”, “id”: |
| had past? | “past” | “schemas”: { | “http://snomed.info/sct/4009004”, “type”: |
| any UTI | | “searchSize”: 10, | “code”, “provenanceType”: “cerner custom |
| past | | “scoreThreshold”: 0.0, | synonym”, “score”: 1.0, “code”: “4009004”, |
| | | “searchMethod”: | “system”: “SNOMEDCT”, “display”: “Lower urinary |
| | | “boolean”, | tract infectious disease”, “semanticType”: [ |
| | | “normalizationKnnMax”: | “disorder” ] }, { “nl”: “uti”, “id”: |
| | | 15.0, | “http://snomed.info/sct/68566005”, “type”: |
| | | “booleanSearchConfig”: | “code”, “provenanceType”: “cerner custom |
| | | { “knnK”: 200, | synonym”, “score”: 1.0, “code”: “68566005”, |
| | | “matchWeight”: 0.0, | “system”: “SNOMEDCT”, “display”: “Urinary tract |
| | | “knnWeight”: 1.0 }, | infectious disease”, “semanticType”: [ “disorder” ] }, |
| | | “embeddingMethod”: | { “nl”: “bacterial uti (urinary tract infection)”, “id”: |
| | | “sapbert” }, “codings”: { | “http://snomed.info/sct/312124009”, “type”: |
| | | “searchSize”: 20, | “code”, “provenanceType”: “name”, “score”: 0.839- |
| | | “scoreThreshold”: 0.4, | 890-0869565217, “code”: “312124009”, “system”: |
| | | “searchMethod”: | “SNOMEDCT”, “display”: “Bacterial urinary |
| | | “boolean”, | infection”, “semanticType”: [ “disorder” ] }, { “nl”: |
| | | “normalizationKnnMax”: | “acute uti”, “id”: |
| | | 23.0, | “http://snomed.info/sct/431309003”, “type”: |
| | | “booleanSearchConfig”: | “code”, “provenanceType”: “cerner custom |
| | | { “knnK”: 200, | synonym”, “score”: 0.812-112-2173913043, |
| | | “matchWeight”: 0.0, | “code”: “431309003”, “system”: “SNOMEDCT”, |
| | | “knnWeight”: 1.0 }, | “display”: “Acute urinary tract infection (disorder)”, |
| | | “embeddingMethod”: | “semanticType”: [ “disorder” ] }, { “nl”: “upper uti”, |
| | | “sapbert”, | “id”: |
| | | “enablePreprocessor”: | “http://snomed.info/sct/422747000”, “type”: |
| | | true, | “code”, “provenanceType”: “cerner custom |
| | | “preprocessMethod”: | synonym”, “score”: 0.808-571-695652174, |
| | | “seg_by_stopwords”, | “code”: “422747000”, “system”: “SNOMEDCT”, |
| | | “codingSystems”: { | “display”: “Upper urinary tract infection (disorder)”, |
| | | “LOINC”: { | “semanticType”: [ “disorder” ] }, { “nl”: “uti |
| | | “searchSize”: 1, | symptoms”, “id”: |
| | | “scoreThreshold”: 0.7, | “http://snomed.info/sct/249274008”, “type”: |
| | | “searchMethod”: | “code”, “provenanceType”: “cerner custom |
| | | “boolean”, | synonym”, “score”: 0.793-324-8260869565, |
| | | “normalizationKnnMax”: | “code”: “249274008”, “system”: “SNOMEDCT”, |
| | | 23.0, | “display”: “Urinary symptoms”, “semanticType”: [ |
| | | “booleanSearchConfig”: | “finding” ] }, { “nl”: “utibid”, “id”: |
| | | { “knnK”: 100, | “http://snomed.info/sct/66653008”, “type”: |
| | | “matchWeight”: 0.2, | “code”, “provenanceType”: “cerner custom |
| | | “knnWeight”: 0.8 } }, | synonym”, “score”: 0.791-395-0869565217, |
| | | “LOINC PART”: { | “code”: “66653008”, “system”: “SNOMEDCT”, |
| | | “searchSize”: 20, | “display”: “Oxolinic acid”, “semanticType”: [ |
| | | “scoreThreshold”: 0.4, | “substance” ] }, { “nl”: “history of past illness”, “id”: |
| | | “searchMethod”: | “http://loinc.org/11349-8”, “type”: “code”, |
| | | “boolean”, | “provenanceType”: “name”, “score”: 0.700-588- |
| | | “normalizationKnnMax”: | 9215686274, “code”: “11349-8”, “system”: |
| | | 23.0, | “LOINC”, “display”: “History of Past illness Nom”, |
| | | “booleanSearchConfig”: | “semanticType”: [ ] }, { “nl”: “past medical history”, |
| | | { “knnK”: 200, | “id”: “http://loinc.org/LP74217-8”, “type”: |
| | | “matchWeight”: 0.0, | “code”, “provenanceType”: “part name”, “score”: |
| | | “knnWeight”: 1.0 } }, | 0.661-257-0434782609, “code”: “LP74217-8”, |
| | | “SNOMEDCT”: { | “system”: “LOINC PART”, “display”: “Past medical |
| | | “searchSize”: 20, | history”, “semanticType”: [ “COMPONENT” ] }, { |
| | | “scoreThreshold”: 0.4, | “nl”: “history”, “id”: “http://loinc.org/LP7800- |
| | | “searchMethod”: | 8”, “type”: “code”, “provenanceType”: “part name”, |
| | | “boolean”, | “score”: 0.549061, “code”: “LP7800-8”, “system”: |
| | | “normalizationKnnMax”: | “LOINC PART”, “display”: “History”, |
| | | 23.0, | “semanticType”: [ “CLASS” ] }, { “nl”: “history”, |
| | | “booleanSearchConfig”: | “id”: “http://loinc.org/LP30639-6”, “type”: |
| | | { “knnK”: 200, | “code”, “provenanceType”: “part name”, “score”: |
| | | “matchWeight”: 0.0, | 0.549061, “code”: “LP30639-6”, “system”: “LOINC |
| | | “knnWeight”: 1.0 } }, | PART”, “display”: “History”, “semanticType”: [ |
| | | “HL7”: { “searchSize”: | “SYSTEM” ] }, { “nl”: “post”, “id”: |
| | | 20, “scoreThreshold”: | “http://loinc.org/LP32606-3”, “type”: “code”, |
| | | 0.4, “searchMethod”: | “provenanceType”: “part name”, “score”: 0.547- |
| | | “boolean”, | 530-1304347826, “code”: “LP32606-3”, “system”: |
| | | “normalizationKnnMax”: | “LOINC PART”, “display”: “post”, “semanticType”: |
| | | 23.0, | [ “COMPONENT” ] }, { “nl”: “past”, “id”: |
| | | “booleanSearchConfig”: | “http://snomed.info/sct/410513005”, “type”: |
| | | { “knnK”: 200, | “code”, “provenanceType”: “name”, “score”: 0.842- |
| | | “matchWeight”: 0.0, | 977-7826086956, “code”: “410513005”, “system”: |
| | | “knnWeight”: 1.0 } } } } | “SNOMEDCT”, “display”: “In the past(qualifier |
| | | } | value)”, “semanticType”: [ “qualifier value” ] }, { |
| | | | “nl”: “past procedure in the past”, “id”: |
| | | | “http://snomed.info/sct/416940007”, “type”: |
| | | | “code”, “provenanceType”: “cerner custom |
| | | | synonym”, “score”: 0.763-752-4782608694, |
| | | | “code”: “416940007”, “system”: “SNOMEDCT”, |
| | | | “display”: “Past history of procedure (context- |
| | | | dependent category)”, “semanticType”: [ “situation” |
| | | | ] } ] }, “debug”: { “config”: { “indexName”: |
| | | | “meaning_index”, “schemas”: { “searchSize”: 10, |
| | | | “scoreThreshold”: 0.0, “searchMethod”: “boolean”, |
| | | | “normalizationKnnMax”: 15.0, |
| | | | “booleanSearchConfig”: { “knnK”: 200, |
| | | | “matchWeight”: 0.0, “knnWeight”: 1.0 }, |
| | | | “embeddingMethod”: “sapbert” }, “codings”: { |
| | | | “searchSize”: 20, “scoreThreshold”: 0.4, |
| | | | “searchMethod”: “boolean”, |
| | | | “normalizationKnnMax”: 23.0, |
| | | | “booleanSearchConfig”: { “knnK”: 200, |
| | | | “matchWeight”: 0.0, “knnWeight”: 1.0 }, |
| | | | “embeddingMethod”: “sapbert”, |
| | | | “enablePreprocessor”: true, “preprocessMethod”: |
| | | | “seg_by_stopwords”, “codingSystems”: { “LOINC”: |
| | | | { “searchSize”: 1, “scoreThreshold”: 0.7, |
| | | | “searchMethod”: “boolean”, |
| | | | “normalizationKnnMax”: 23.0, |
| | | | “booleanSearchConfig”: { “knnK”: 100, |
| | | | “matchWeight”: 0.2, “knnWeight”: 0.8 } }, “LOINC |
| | | | PART”: { “searchSize”: 20, “scoreThreshold”: 0.4, |
| | | | “searchMethod”: “boolean”, |
| | | | “normalizationKnnMax”: 23.0, |
| | | | “booleanSearchConfig”: { “knnK”: 200, |
| | | | “matchWeight”: 0.0, “knnWeight”: 1.0 } }, |
| | | | “SNOMEDCT”: { “searchSize”: 20, |
| | | | “scoreThreshold”: 0.4, “searchMethod”: “boolean”, |
| | | | “normalizationKnnMax”: 23.0, |
| | | | “booleanSearchConfig”: { “knnK”: 200, |
| | | | “matchWeight”: 0.0, “knnWeight”: 1.0 } }, “HL7”: { |
| | | | “searchSize”: 20, “scoreThreshold”: 0.4, |
| | | | “searchMethod”: “boolean”, |
| | | | “normalizationKnnMax”: 23.0, |
| | | | “booleanSearchConfig”: { “knnK”: 200, |
| | | | “matchWeight”: 0.0, “knnWeight”: 1.0 } } } } }, |
| | | | “latency”: { “preprocess”: “Process took 20.44ms”, |
| | | | “embedding”: “Process took 42.37ms”, |
| | | | “search.opensearch”: “Process took 160.58 ms”, |
| | | | “search”: “Process took 136 ms”, “overall”: “Process |
| | | | took 255.46 ms” }, “segments”: [ “UTI”, “past” ] } } |

The link to concepts API pipeline 415 represents a second, more targeted real-time process designed for high-precision entity normalization. This pipeline is initiated when a structured request, typically from an upstream system that has already performed Named Entity Recognition (NER), is received at a dedicated API endpoint 475. The request payload contains one or more pre-identified entities, where each entity is a structured object comprising an identifier, a specific text value (e.g., “blood pressure”), and an associated entity type (e.g., “OBSERVATION”). The request may also specify a list of target coding systems to constrain the search, such as SNOMEDCT or LOINC.

For each individual entity contained within the request, an embedding generation module 435 is invoked. This module passes the entity's text to the pre-trained semantic embedding model 440 (e.g., SapBert) to generate a high-dimensional entity vector that numerically represents the entity's precise clinical meaning. This process is repeated for every entity in the request payload, creating a set of distinct vectors to be linked.

These entity vectors, along with a defined search configuration, are then processed by the hybrid search module 465. The module executes a highly constrained and targeted query for each vector against the vector database 420 using the similarity search engine 470. The search is intelligently filtered based on the metadata provided in the initial request; for instance, the entity's type is used to narrow the search space to only relevant concept categories, and the list of codingSystems further restricts the search to specific terminologies. This multi-layered filtering ensures high precision and relevance. The underlying search mechanism remains a hybrid of k-Nearest Neighbors (kNN) vector search for semantic closeness (e.g., based on semantic proximity) and keyword search for lexical matching.

The hybrid search module 465 retrieves the top-scoring matched concepts for each input entity and ranks these results in descending order based on their computed confidence scores. The module then constructs a detailed, structured response that explicitly links each original entity, using its identifier, to its corresponding ranked list of matched concepts. This list may be organized by coding system and includes the formal codes, display names, and confidence scores for each potential match. This structured response is then returned to the calling system via the API endpoint 480, providing a precise, prioritized, and machine-readable annotation of the original clinical entities with minimal latency, thereby allowing downstream applications to intelligently select the most appropriate concept based on the provided ranking.

To provide a practical illustration of the link to concepts API pipeline 415, the following Table 3 demonstrates the end-to-end process for a specific, pre-identified entity. The example shows the pipeline receiving an entity with its text and type, computing its vector embedding, applying a specific search configuration, and ultimately producing a structured JSON response. This response explicitly links the original entity (“blood pressure”) to a ranked list of matched concepts from multiple coding systems (SNOMEDCT and LOINC), complete with their official codes, display names, and confidence scores. This showcases the pipeline's ability to deliver precise, targeted, and context-aware concept normalization for structured inputs.

TABLE 3

| Entity in Text | search config | Entity with Linked Concepts |
|---|---|---|
| json { “text”: “blood | json { “key”: “key”, | json { “key”: “key”, “entities”: [{ “id”: “e1”, |
| pressure”, “type”: | “entities”: [{ “id”: “e1”, | “matchedConcepts”: [{ “codingSystem”: |
| “OBSERVATION” } | “text”: “blood pressure”, | “SNOMEDCT”, “concepts”: [ { “code”: “773093006”, |
| | “type”: “OBSERVATION” | “display”: “ed note”, “score”: 1.0, “attributes”: { |
| | }], “codingSystems”: [ | “semantic_type”: “Doc” } }, { “code”: “96335-5”, |
| | “RXNORM”, | “display”: “ED record artifact”, “score”: 0.7901, |
| | “SNOMEDCT”, | “attributes”: { “semantic_type”: “Doc” } }, { “code”: |
| | “ICD10CM”, “LOINC” ], | “97669-6”, “display”: “Emergency med ED Prog note”, |
| | “languageCode”: “en” } | “score”: 0.767, “attributes”: { “semantic_type”: “Doc” } |
| | | }, { “code”: “97660-5”, “display”: “Emergency med ED |
| | | Eval note”, “score”: 0.7613, “attributes”: { |
| | | “semantic_type”: “Doc” } }, { “code”: “51848-0”, |
| | | “display”: “ED Eval note”, “score”: 0.7581, “attributes”: |
| | | { “semantic_type”: “Doc” } }, { “code”: “99492-1”, |
| | | “display”: “Geri med ED Eval note”, “score”: 0.7245, |
| | | “attributes”: { “semantic_type”: “Doc” } }, {“ code”: |
| | | “97665-4”, “display”: “Emergency med ED Educ note”, |
| | | “score”: 0.7209, “attributes”: { “semantic_type”: “Doc” |
| | | } } ] }, { “codingSystem”: “LOINC”, “concepts”: [ { |
| | | “code”: “34111-5”, “display”: “ED Note”, “score”: 1.0, |
| | | “attributes”: { “semantic_type”: “Note” } }, { “code”: |
| | | “28568-4”, “display”: “MD ED Summary note”, |
| | | “score”: 0.8468, “attributes”: { “semantic_type”: “Doc” |
| | | } } ] }] }], “languageCode”: “en” } |

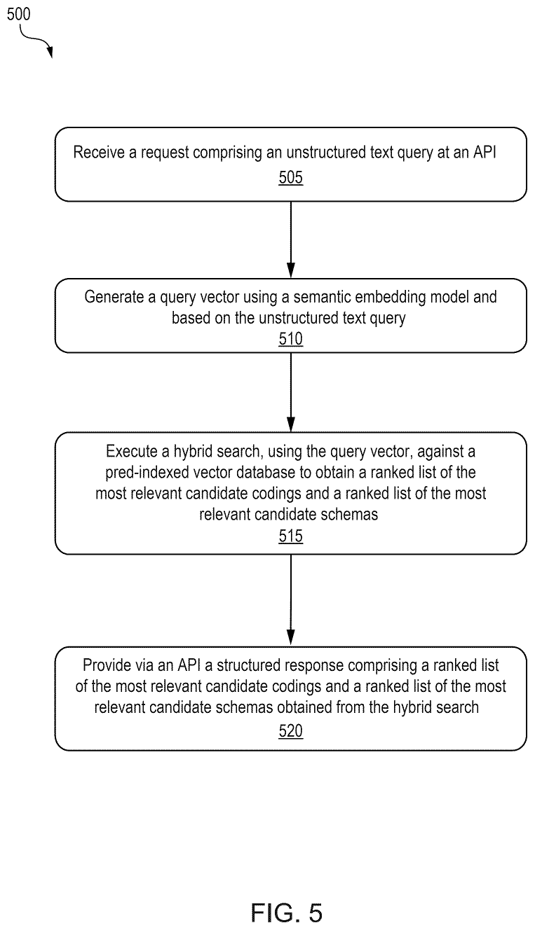

FIG. 5 presents a process flow 500 for linking natural language text to medical codes using a search meaning candidates process. The processing depicted in FIG. 5 may be implemented in software (e.g., code, instructions, a program) executed by one or more processing units (e.g., one or more processors, cores) of the respective systems, hardware, or combinations thereof described throughout. The software may be stored on a non-transitory storage medium (e.g., on a memory device). Although the methods presented in FIG. 5 depict the various processing steps occurring in a particular sequence or order, this is not intended to be limiting. In certain alternative embodiments, the steps may be performed in parallel and/or in a different order. In certain embodiments, such as in the embodiments depicted in FIGS. 1-4, the processing depicted in FIG. 5 may be performed by one or more components of the healthcare environment 100, the digital assistant environment 200, and/or the computing environment 400, as described with respect to FIGS. 1-4.

At step 505, a request comprising an unstructured text query is received at an application programming interface endpoint. The unstructured text query may originate from a user-facing application (such as a digital assistant, clinical chatbot, or EHR interface), an automated workflow, or a third-party system integrating via RESTful API or similar protocol. The API endpoint can support multiple authentication and authorization mechanisms (e.g., OAuth, API keys, JWTs) to ensure secure and auditable access. The request may include metadata such as user ID, session context, language code, or tenant identifier, enabling personalized or context-specific processing.

At step 510, a query vector is generated using a semantic embedding model and based on the unstructured text query (e.g., the unstructured text query is used as input into the semantic embedding model). The query vector represents a semantic meaning of the unstructured text query.

The semantic embedding model may be selected dynamically based on the request context or configuration, supporting alternative models (e.g., SapBERT, BioBERT, ClinicalBERT) for specialized vocabularies or languages. The embedding process may include normalization steps such as lowercasing, accent removal, or Unicode normalization for consistent results. The query vector may be cached for identical or similar queries to reduce redundant computation and improve latency.

In some instances, prior to generating the query vector, the unstructured text query is preprocessed by performing key phrase extraction to identify one or more clinical terms in the unstructured text query. The query vector is generated using the semantic embedding model and based on the one or more clinical terms.

Key phrase extraction may utilize trained NLP models, such as named entity recognition (NER), part-of-speech tagging, or dependency parsing, to more accurately identify multi-word terms and relationships. Stop-word removal, lemmatization, or tokenization may be applied to reduce noise and improve the semantic signal. The system may flag potential PHI in the query and either redact, mask, or encrypt it in accordance with HIPAA or other privacy regulations. Preprocessing results (e.g., extracted key phrases, detected entities, or de-identified text) may be included in the response for transparency or debugging.

At step 515, a hybrid search is executed, using the query vector, against a pre-indexed vector database. The pre-indexed vector database comprises vector representations for medical codings and semantic object schemas, and the hybrid search combines a vector similarity search with a keyword search to retrieve a list of most relevant candidate codings and a list of most relevant candidate schemas.

In the context of the present disclosure, the term “most relevant” refers to a quantifiable measure of the semantic and/or lexical correspondence between an input text query and a candidate concept (either a medical coding or a semantic schema) stored within the vector database. The determination of relevance is the output of a hybrid search process that combines two distinct algorithmic approaches: a vector similarity search and a keyword search.

The vector similarity search determines relevance based on semantic proximity in a high-dimensional vector space. A medical coding is considered “most relevant” if its pre-computed high-dimensional vector representation has the highest calculated similarity score (or smallest distance) relative to the query vector generated from the user's unstructured text. This score, which may be calculated using metrics such as Cosine Similarity or Euclidean Distance, signifies that the clinical meaning or context of the coding is semantically closest to the meaning and context of the input query, even if the literal text does not match. For example, the query “heart attack” would be most relevant to the SNOMED CT code for “Myocardial Infarction” because their vector representations are in close proximity. A semantic object schema is considered “most relevant” if its pre-computed high-dimensional vector representation is semantically closest to the query vector. This indicates that the overall intent or structure of the user's query aligns best with the purpose and attributes of that schema. For example, the query “schedule a follow-up for the patient next Tuesday” would be most relevant to a schema for “Appointment Scheduling” due to the high semantic similarity of their respective vectors.

The keyword search determines relevance based on lexical correspondence between the text of the input query and the textual metadata associated with a candidate concept. A medical coding is considered “most relevant” if its associated textual metadata (which may include its official name, display text, synonyms, abbreviations, and acronyms) has the highest lexical match score with the terms in the input query. This score may be calculated using information retrieval algorithms such as BM25 or TF-IDF, which evaluate the frequency and specificity of matching terms. This ensures that concepts with direct textual overlap, including well-known clinical acronyms (e.g., “UTI”), are ranked highly. A semantic object schema is considered “most relevant” if its associated textual metadata (including its name, description, and the names of its attributes) has the highest lexical match score with the terms in the input query. This allows the system to identify schemas that are explicitly mentioned or described in the user's request.

The final determination of the “most relevant” candidate codings and schemas is achieved by combining the scores from both the vector similarity search and the keyword search, for example, through a configurable weighted formula. This hybrid approach ensures that the ultimate ranking reflects both deep semantic understanding and precise lexical matching, providing a comprehensive and accurate measure of relevance.

In some instances, the vector database comprises a plurality of indexed vectors corresponding to a plurality of standardized medical codings and a plurality of semantic object schemas. The plurality of standardized medical codings may be from one or more of: Systematized Nomenclature of Medicine-Clinical Terms (SNOMED CT), International Classification of Diseases (ICD), Logical Observation Identifiers Names and Codes (LOINC), or RxNorm.

The vector database may periodically synchronize with upstream coding system releases to ensure up-to-date concept coverage and accuracy. Semantic object schemas in the database may include hierarchical or compositional relationships (e.g., parent-child, attribute-level mappings) and can be tenant-agnostic or tenant-specific as required. The vector database may be partitioned by coding system, semantic type, or tenant, allowing for efficient sharding and horizontal scaling.

In some instances, the vector similarity search is a k-Nearest Neighbors (kNN) vector similarity search that is executed based on the query vector to identify a first set of results, the keyword search is executed based on the query vector to identify a second set of results, and the first and second sets of results are combined based on a weight for the vector similarity search and a weight for the key word search to retrieve the list of most relevant candidate codings and the list of most relevant candidate schemas.

The hybrid search may also support additional search modes, such as faceted filtering (e.g., limit results to a specific clinical specialty or date range) or semantic boosting (e.g., prioritize recent or commonly used codes). The search engine may employ approximate nearest neighbor (ANN) search algorithms (e.g., HNSW, FAISS, Annoy) for sublinear time performance on very large indexes. Search results may include auxiliary metadata, such as code validity period, deprecation status, or mapping to legacy coding systems (e.g., ICD-9). Data consistency and integrity checks (e.g., referential integrity, schema validation) may be performed during indexing and search.

The weighting of kNN and keyword search results may be configurable per query or per client, allowing for fine-tuned balancing of semantic and lexical relevance. The system may support fallback logic: for queries with ambiguous or low-confidence matches, the system may return additional candidates or request disambiguation from the calling application. The system may log search queries, configurations, and outcomes for monitoring, analytics, and continuous improvement of model accuracy.

In some instances, the request further comprises a search configuration, and executing the hybrid search is governed by one or more parameters in the search configuration, the one or more parameters comprising a search size, a score threshold, a weight for the vector similarity search, a weight for the key word search, or any combination thereof.

The search configuration can include advanced features such as: Semantic type filtering: e.g., restrict results to diagnoses, medications, or lab tests; Contextual weighting: e.g., increase the score for codes frequently used by the requesting clinician or department; and Custom synonym dictionaries: enable tenant-specific or organization-specific synonyms to be considered in the search. Default configuration values may be set at the system, tenant, or user level, with override capabilities per request.

At step 520, a structured response is provided via the application programming interface (in some instances this may be a different application programming interface from that in which the response was received). The structured responses comprises a ranked list of the most relevant candidate codings and a ranked list of the most relevant candidate schemas.

The ranked lists of candidate codings and schemas may include not only scores but also explanation fields (e.g., why a particular code was matched, which features or terms contributed most to the match). The response format may support pagination, allowing the client to request additional candidates as needed. The application programming interface may provide versioning to ensure backward compatibility as schema or scoring logic evolves.

In some instances, the structured response is configured for consumption by a downstream schema synthesizer configured to construct a machine-readable representation of the unstructured text query. The downstream schema synthesizer may generate complex query plans, database queries (e.g., SQL, FHIRPath), or even logic for orchestration of automated workflows using the normalized schema.

In some instances, each of the most relevant candidate codings in the structured response includes a unique identifier, a display name, a source coding system, and a similarity score.

In some instances, the process further comprises: generating, by one or more generative models based on the unstructured text query and the structured response, an execution plan that identifies one or more agents to execute one or more actions or operations responsive to the unstructured text query and the structured response; executing, by the one or more agents, the one or more actions or operations on one or more assets and/or a context and memory store, where the execution of the one or more actions or operations generates output data from the one or more assets and/or relevant context and memory information from the context and memory store; generating, by one or more generative models based on an output prompt, a response to the unstructured text query, where the output prompt comprises the output data and/or the relevant context and the memory information; and providing the response to the unstructured text query. The response to the unstructured query may be provided through multiple modalities, such as structured data payloads, human-readable summaries, or even voice or text-based interfaces.

In some instances, the process further comprises: generating, by one or more generative models based on an output prompt, a response to the unstructured text query, wherein the output prompt comprises the unstructured text query and/or the ranked list of the most relevant candidate codings and the ranked list of the most relevant candidate schemas; and providing the response to the unstructured text query. The response to the unstructured query may be provided through multiple modalities, such as structured data payloads, human-readable summaries, or even voice or text-based interfaces.

As should be understood, the one or more generative models described with respect to process flow 500 may all be the same or different generative models; or a subset of the one or more generative models may be the same generative models while another subset of the one or more generative models may be different generative models. For example, the one or more generative models that generate a response to the unstructured text query may be the same or different generative models from the one or more generative models that generate the execution plan. In instances, where the one or more generative models are the same generative models, the system would make multiple calls to the same one or more generative models (e.g., a call to initiate generation of the execution plan and separate call to initiate generation of the response).

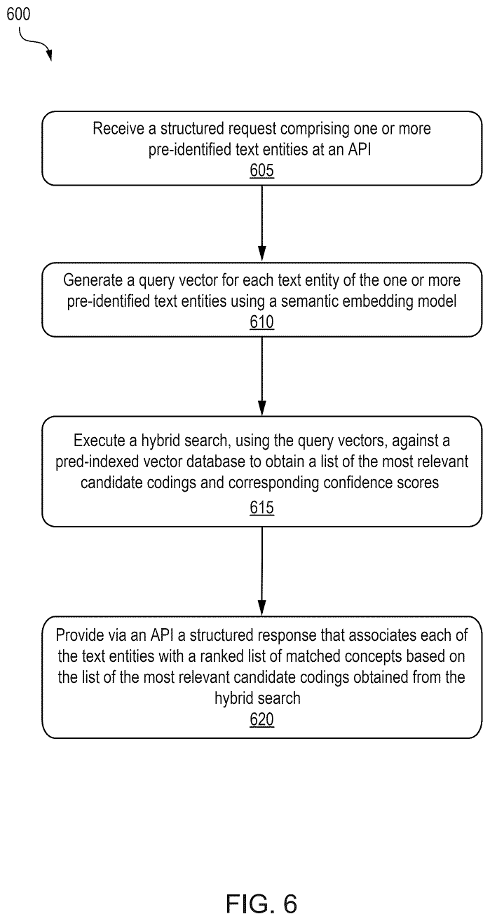

FIG. 6 presents a process flow 600 for linking specific clinical entities to medical codes using a link to concept process. The processing depicted in FIG. 6 may be implemented in software (e.g., code, instructions, a program) executed by one or more processing units (e.g., one or more processors, cores) of the respective systems, hardware, or combinations thereof described throughout. The software may be stored on a non-transitory storage medium (e.g., on a memory device). Although the methods presented in FIG. 6 depict the various processing steps occurring in a particular sequence or order, this is not intended to be limiting. In certain alternative embodiments, the steps may be performed in parallel and/or in a different order. In certain embodiments, such as in the embodiments depicted in FIGS. 1-4, the processing depicted in FIG. 6 may be performed by one or more components of the healthcare environment 100, the digital assistant environment 200, and/or the computing environment 400, as described with respect to FIGS. 1-4.

At step 605, a structured request comprising one or more pre-identified text entities is received at an application programming interface endpoint. Each text entity of the one or more pre-identified text entities may have an associated entity type. The structured request may originate from a user-facing application (such as a digital assistant, clinical chatbot, or EHR interface), an automated workflow, or a third-party system integrating via RESTful API or similar protocol. The API endpoint can support multiple authentication and authorization mechanisms (e.g., OAuth, API keys, JWTs) to ensure secure and auditable access. The request may include metadata such as user ID, session context, language code, or tenant identifier, enabling personalized or context-specific processing.

In some instances, the one or more identified text entities in the structured request are generated by a Named Entity Recognition (NER) process applied to a natural language utterance.

At step 610, for each text entity, a query vector representing a semantic meaning of each entity is generated using a semantic embedding model and based on the structured request.

The semantic embedding model may be selected dynamically based on the request context or configuration, supporting alternative models (e.g., SapBERT, BioBERT, ClinicalBERT) for specialized vocabularies or languages. The embedding process may include normalization steps such as lowercasing, accent removal, or Unicode normalization for consistent results. The query vector may be cached for identical or similar queries to reduce redundant computation and improve latency.

At step 615, a hybrid search is executed, using the query vector for each text entity, against a pre-indexed vector database. The pre-indexed vector database comprises vector representations for medical codings, the hybrid search combines a vector similarity search with a keyword search to retrieve a list of most relevant candidate codings and corresponding confidence scores. The hybrid search may be constrained by the entity type associated with the text entity.

As described herein, in the context of the present disclosure, the term “most relevant” refers to a quantifiable measure of the semantic and/or lexical correspondence between an input text query (in this instance the text entity) and a candidate concept (either a medical coding or a semantic schema) stored within the vector database. The determination of relevance is the output of a hybrid search process that combines two distinct algorithmic approaches: a vector similarity search and a keyword search.

The vector similarity search determines relevance based on semantic proximity in a high-dimensional vector space. A medical coding is considered “most relevant” if its pre-computed high-dimensional vector representation has the highest calculated similarity score (or smallest distance) relative to the query vector generated from the text entity. This score, which may be calculated using metrics such as Cosine Similarity or Euclidean Distance, signifies that the clinical meaning or context of the coding is semantically closest to the meaning and context of the text entity, even if the literal text does not match. For example, the text entity “heart attack” would be most relevant to the SNOMED CT code for “Myocardial Infarction” because their vector representations are in close proximity.

The keyword search determines relevance based on lexical correspondence between the text entity and the textual metadata associated with a candidate concept. A medical coding is considered “most relevant” if its associated textual metadata (which may include its official name, display text, synonyms, abbreviations, and acronyms) has the highest lexical match score with the text entity. This score may be calculated using information retrieval algorithms such as BM25 or TF-IDF, which evaluate the frequency and specificity of matching terms. This ensures that concepts with direct textual overlap, including well-known clinical acronyms (e.g., “UTI”), are ranked highly.

The final determination of the “most relevant” candidate codings is achieved by combining the scores from both the vector similarity search and the keyword search, for example, through a configurable weighted formula. This hybrid approach ensures that the ultimate ranking reflects both deep semantic understanding and precise lexical matching, providing a comprehensive and accurate measure of relevance.

In some instances, the vector database comprises a plurality of indexed vectors corresponding to a plurality of standardized medical codings and a plurality of semantic object schemas. The plurality of standardized medical codings may be from one or more of: Systematized Nomenclature of Medicine-Clinical Terms (SNOMED CT), International Classification of Diseases (ICD), Logical Observation Identifiers Names and Codes (LOINC), or RxNorm.

The vector database may periodically synchronize with upstream coding system releases to ensure up-to-date concept coverage and accuracy. Semantic object schemas in the database may include hierarchical or compositional relationships (e.g., parent-child, attribute-level mappings) and can be tenant-agnostic or tenant-specific as required. The vector database may be partitioned by coding system, semantic type, or tenant, allowing for efficient sharding and horizontal scaling.

In some instances, the vector similarity search is a k-Nearest Neighbors (kNN) vector similarity search that is executed based on the query vector to identify a first set of results, the keyword search is executed based on the query vector to identify a second set of results, and the first and second sets of results are combined based on a weight for the vector similarity search and a weight for the key word search to retrieve the list of most relevant candidate codings.

The ranking algorithm may combine similarity scores from kNN and keyword search with heuristic or learned weights, configurable per use case or client. Confidence scores may be calibrated using statistical normalization, model uncertainty estimation, or learned thresholds from historical data. The ranking process may provide explanations or provenance metadata for each match (e.g., “matched via synonym X,” “highest cosine similarity”), improving transparency for downstream users.

In some instances, constraining the hybrid search based on the entity type associated with the text entity comprises searching only a subset of the pre-indexed vector database that corresponds to concepts associated with the entity type.

In some instances, the structured request further comprises a list of one or more target coding systems, and the hybrid search for each text entity is further constrained to concepts belonging to the one or more target coding systems.

Constraints may be dynamically adjusted based on detected ambiguity or user preferences (e.g., allow broader search if initial results are low-confidence). The system may automatically suggest alternative entity types or coding systems if no relevant matches are found. Custom tenant or organization-specific code sets may be incorporated alongside public coding systems, supporting proprietary workflows.

At step 620, a structured response is provided via the application programming interface. The structured response associates each of the text entities with a ranked list of matched concepts based on the list of most relevant candidate codings and the corresponding confidence scores.

In some instances, the process further comprises: generating, by one or more generative models based on a natural language utterance, an execution plan that identifies one or more agents to execute one or more actions or operations responsive to the natural language utterance; executing, by the one or more agents, the one or more actions or operations on one or more assets and/or a context and memory store, wherein the execution of the one or more actions or operations generates output data from the one or more assets and/or relevant context and memory information from the context and memory store; generating, by one or more generative models based on an output prompt, a response to the natural language utterance, wherein the output prompt comprises: (i) the output data and/or the relevant context and the memory information, and (ii) the structured response that associates each of the text entities with the ranked list of matched concepts; and providing the response to the natural language utterance. The response to the natural language utterance may be provided through multiple modalities, such as structured data payloads, human-readable summaries, or even voice or text-based interfaces.

In some instances, the process further comprises: generating, by one or more generative models based on an output prompt, a response to a natural language utterance, wherein the output prompt comprises: (i) the natural language utterance, and (ii) the structured response that associates each of the text entities with a ranked list of matched concepts; and providing the response to the natural language utterance. The response to the natural language utterance may be provided through multiple modalities, such as structured data payloads, human-readable summaries, or even voice or text-based interfaces.

In some instances, the process further comprises: searching, using the structured response that associates each of the text entities with the ranked list of matched concepts, a semantic knowledge graph to identify one or more relationships between text entities; generating, by one or more generative models based on an output prompt, a response to a natural language utterance, wherein the output prompt comprises: (i) the natural language utterance, (ii) the structured response that associates each of the text entities with the ranked list of matched concepts, and the (iii) the one or more relationships between the text entities; and providing the response to the natural language utterance. The response to the natural language utterance may be provided through multiple modalities, such as structured data payloads, human-readable summaries, or even voice or text-based interfaces.

As should be understood, the one or more generative models described with respect to process flow 600 may all be the same or different generative models; or a subset of the one or more generative models may be the same generative models while another subset of the one or more generative models may be different generative models. For example, the one or more generative models that generate a response to the unstructured text query may be the same or different generative models from the one or more generative models that generate the execution plan. In instances, where the one or more generative models are the same generative models, the system would make multiple calls to the same one or more generative models (e.g., a call to intiate generation of the execution plan and separate call to intiate generation of the response).

### Example Cloud Service Provider Infrastructure (CSPI) Architectures

As noted above, infrastructure as a service (IaaS) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an laaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 7 is a block diagram 700 illustrating an example pattern of an IaaS architecture, according to at least one embodiment. Service operators 702 can be communicatively coupled to a secure host tenancy 704 that can include a virtual cloud network (VCN) 706 and a secure host subnet 708. In some examples, the service operators 702 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 706 and/or the Internet.

The VCN 706 can include a local peering gateway (LPG) 710 that can be communicatively coupled to a secure shell (SSH) VCN 712 via an LPG 710 contained in the SSH VCN 712. The SSH VCN 712 can include an SSH subnet 714, and the SSH VCN 712 can be communicatively coupled to a control plane VCN 716 via the LPG 710 contained in the control plane VCN 716. Also, the SSH VCN 712 can be communicatively coupled to a data plane VCN 718 via an LPG 710. The control plane VCN 716 and the data plane VCN 718 can be contained in a service tenancy 719 that can be owned and/or operated by the IaaS provider.

The control plane VCN 716 can include a control plane demilitarized zone (DMZ) tier 720 that acts as a perimeter network (e.g., portions of a corporate network between the corporate intranet and external networks). The DMZ-based servers may have restricted responsibilities and help keep breaches contained. Additionally, the DMZ tier 720 can include one or more load balancer (LB) subnet(s) 722, a control plane app tier 724 that can include app subnet(s) 726, a control plane data tier 728 that can include database (DB) subnet(s) 730 (e.g., frontend DB subnet(s) and/or backend DB subnet(s)). The LB subnet(s) 722 contained in the control plane DMZ tier 720 can be communicatively coupled to the app subnet(s) 726 contained in the control plane app tier 724 and an Internet gateway 734 that can be contained in the control plane VCN 716, and the app subnet(s) 726 can be communicatively coupled to the DB subnet(s) 730 contained in the control plane data tier 728 and a service gateway 736 and a network address translation (NAT) gateway 738. The control plane VCN 716 can include the service gateway 736 and the NAT gateway 738.

The control plane VCN 716 can include a data plane mirror app tier 740 that can include app subnet(s) 726. The app subnet(s) 726 contained in the data plane mirror app tier 740 can include a virtual network interface controller (VNIC) 742 that can execute a compute instance 744. The compute instance 744 can communicatively couple the app subnet(s) 726 of the data plane mirror app tier 740 to app subnet(s) 726 that can be contained in a data plane app tier 746.

The data plane VCN 718 can include the data plane app tier 746, a data plane DMZ tier 748, and a data plane data tier 750. The data plane DMZ tier 748 can include LB subnet(s) 722 that can be communicatively coupled to the app subnet(s) 726 of the data plane app tier 746 and the Internet gateway 734 of the data plane VCN 718. The app subnet(s) 726 can be communicatively coupled to the service gateway 736 of the data plane VCN 718 and the NAT gateway 738 of the data plane VCN 718. The data plane data tier 750 can also include the DB subnet(s) 730 that can be communicatively coupled to the app subnet(s) 726 of the data plane app tier 746.

The Internet gateway 734 of the control plane VCN 716 and of the data plane VCN 718 can be communicatively coupled to a metadata management service 752 that can be communicatively coupled to public Internet 754. Public Internet 754 can be communicatively coupled to the NAT gateway 738 of the control plane VCN 716 and of the data plane VCN 718. The service gateway 736 of the control plane VCN 716 and of the data plane VCN 718 can be communicatively coupled to cloud services 756.

In some examples, the service gateway 736 of the control plane VCN 716 or of the data plane VCN 718 can make application programming interface (API) calls to cloud services 756 without going through public Internet 754. The API calls to cloud services 756 from the service gateway 736 can be one-way: the service gateway 736 can make API calls to cloud services 756, and cloud services 756 can send requested data to the service gateway 736. But, cloud services 756 may not initiate API calls to the service gateway 736.

In some examples, the secure host tenancy 704 can be directly connected to the service tenancy 719, which may be otherwise isolated. The secure host subnet 708 can communicate with the SSH subnet 714 through an LPG 710 that may enable two-way communication over an otherwise isolated system. Connecting the secure host subnet 708 to the SSH subnet 714 may give the secure host subnet 708 access to other entities within the service tenancy 719.

The control plane VCN 716 may allow users of the service tenancy 719 to set up or otherwise provision desired resources. Desired resources provisioned in the control plane VCN 716 may be deployed or otherwise used in the data plane VCN 718. In some examples, the control plane VCN 716 can be isolated from the data plane VCN 718, and the data plane mirror app tier 740 of the control plane VCN 716 can communicate with the data plane app tier 746 of the data plane VCN 718 via VNICs 742 that can be contained in the data plane mirror app tier 740 and the data plane app tier 746.

In some examples, users of the system, or customers, can make requests, for example create, read, update, or delete (CRUD) operations, through public Internet 754 that can communicate the requests to the metadata management service 752. The metadata management service 752 can communicate the request to the control plane VCN 716 through the Internet gateway 734. The request can be received by the LB subnet(s) 722 contained in the control plane DMZ tier 720. The LB subnet(s) 722 may determine that the request is valid, and in response to this determination, the LB subnet(s) 722 can transmit the request to app subnet(s) 726 contained in the control plane app tier 724. If the request is validated and requires a call to public Internet 754, the call to public Internet 754 may be transmitted to the NAT gateway 738 that can make the call to public Internet 754. Metadata that may be desired to be stored by the request can be stored in the DB subnet(s) 730.

In some examples, the data plane mirror app tier 740 can facilitate direct communication between the control plane VCN 716 and the data plane VCN 718. For example, changes, updates, or other suitable modifications to configuration may be desired to be applied to the resources contained in the data plane VCN 718. Via a VNIC 742, the control plane VCN 716 can directly communicate with, and can thereby execute the changes, updates, or other suitable modifications to configuration to, resources contained in the data plane VCN 718.

In some embodiments, the control plane VCN 716 and the data plane VCN 718 can be contained in the service tenancy 719. In this case, the user, or the customer, of the system may not own or operate either the control plane VCN 716 or the data plane VCN 718. Instead, the IaaS provider may own or operate the control plane VCN 716 and the data plane VCN 718, both of which may be contained in the service tenancy 719. This embodiment can enable isolation of networks that may prevent users or customers from interacting with other users', or other customers', resources. Also, this embodiment may allow users or customers of the system to store databases privately without needing to rely on public Internet 754, which may not have a desired level of threat prevention, for storage.

In other embodiments, the LB subnet(s) 722 contained in the control plane VCN 716 can be configured to receive a signal from the service gateway 736. In this embodiment, the control plane VCN 716 and the data plane VCN 718 may be configured to be called by a customer of the IaaS provider without calling public Internet 754. Customers of the IaaS provider may desire this embodiment since database(s) that the customers use may be controlled by the IaaS provider and may be stored on the service tenancy 719, which may be isolated from public Internet 754.

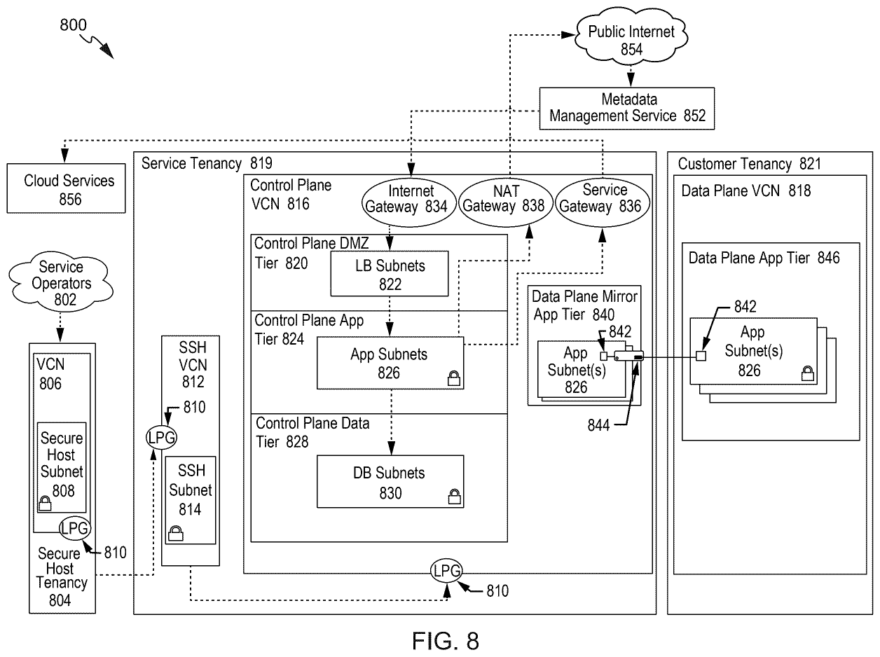

FIG. 8 is a block diagram 800 illustrating another example pattern of an IaaS architecture, according to at least one embodiment. Service operators 802 (e.g., service operators 702 of FIG. 7) can be communicatively coupled to a secure host tenancy 804 (e.g., the secure host tenancy 704 of FIG. 7) that can include a virtual cloud network (VCN) 806 (e.g., the VCN 706 of FIG. 7) and a secure host subnet 808 (e.g., the secure host subnet 708 of FIG. 7). The VCN 806 can include a local peering gateway (LPG) 810 (e.g., the LPG 710 of FIG. 7) that can be communicatively coupled to a secure shell (SSH) VCN 812 (e.g., the SSH VCN 712 of FIG. 7) via an LPG 710 contained in the SSH VCN 812. The SSH VCN 812 can include an SSH subnet 814 (e.g., the SSH subnet 714 of FIG. 7), and the SSH VCN 812 can be communicatively coupled to a control plane VCN 816 (e.g., the control plane VCN 716 of FIG. 7) via an LPG 810 contained in the control plane VCN 816. The control plane VCN 816 can be contained in a service tenancy 819 (e.g., the service tenancy 719 of FIG. 7), and the data plane VCN 818 (e.g., the data plane VCN 718 of FIG. 7) can be contained in a customer tenancy 821 that may be owned or operated by users, or customers, of the system.

The control plane VCN 816 can include a control plane DMZ tier 820 (e.g., the control plane DMZ tier 720 of FIG. 7) that can include LB subnet(s) 822 (e.g., LB subnet(s) 722 of FIG. 7), a control plane app tier 824 (e.g., the control plane app tier 724 of FIG. 7) that can include app subnet(s) 826 (e.g., app subnet(s) 726 of FIG. 7), a control plane data tier 828 (e.g., the control plane data tier 728 of FIG. 7) that can include database (DB) subnet(s) 830 (e.g., similar to DB subnet(s) 730 of FIG. 7). The LB subnet(s) 822 contained in the control plane DMZ tier 820 can be communicatively coupled to the app subnet(s) 826 contained in the control plane app tier 824 and an Internet gateway 834 (e.g., the Internet gateway 734 of FIG. 7) that can be contained in the control plane VCN 816, and the app subnet(s) 826 can be communicatively coupled to the DB subnet(s) 830 contained in the control plane data tier 828 and a service gateway 836 (e.g., the service gateway 736 of FIG. 7) and a network address translation (NAT) gateway 838 (e.g., the NAT gateway 738 of FIG. 7). The control plane VCN 816 can include the service gateway 836 and the NAT gateway 838.

The control plane VCN 816 can include a data plane mirror app tier 840 (e.g., the data plane mirror app tier 740 of FIG. 7) that can include app subnet(s) 826. The app subnet(s) 826 contained in the data plane mirror app tier 840 can include a virtual network interface controller (VNIC) 842 (e.g., the VNIC of 742) that can execute a compute instance 844 (e.g., similar to the compute instance 744 of FIG. 7). The compute instance 844 can facilitate communication between the app subnet(s) 826 of the data plane mirror app tier 840 and the app subnet(s) 826 that can be contained in a data plane app tier 846 (e.g., the data plane app tier 746 of FIG. 7) via the VNIC 842 contained in the data plane mirror app tier 840 and the VNIC 842 contained in the data plane app tier 846.

The Internet gateway 834 contained in the control plane VCN 816 can be communicatively coupled to a metadata management service 852 (e.g., the metadata management service 752 of FIG. 7) that can be communicatively coupled to public Internet 854 (e.g., public Internet 754 of FIG. 7). Public Internet 854 can be communicatively coupled to the NAT gateway 838 contained in the control plane VCN 816. The service gateway 836 contained in the control plane VCN 816 can be communicatively coupled to cloud services 856 (e.g., cloud services 756 of FIG. 7).

In some examples, the data plane VCN 818 can be contained in the customer tenancy 821. In this case, the IaaS provider may provide the control plane VCN 816 for each customer, and the IaaS provider may, for each customer, set up a unique compute instance 844 that is contained in the service tenancy 819. Each compute instance 844 may allow communication between the control plane VCN 816, contained in the service tenancy 819, and the data plane VCN 818 that is contained in the customer tenancy 821. The compute instance 844 may allow resources, that are provisioned in the control plane VCN 816 that is contained in the service tenancy 819, to be deployed or otherwise used in the data plane VCN 818 that is contained in the customer tenancy 821.

In other examples, the customer of the IaaS provider may have databases that live in the customer tenancy 821. In this example, the control plane VCN 816 can include the data plane mirror app tier 840 that can include app subnet(s) 826. The data plane mirror app tier 840 can reside in the data plane VCN 818, but the data plane mirror app tier 840 may not live in the data plane VCN 818. That is, the data plane mirror app tier 840 may have access to the customer tenancy 821, but the data plane mirror app tier 840 may not exist in the data plane VCN 818 or be owned or operated by the customer of the IaaS provider. The data plane mirror app tier 840 may be configured to make calls to the data plane VCN 818 but may not be configured to make calls to any entity contained in the control plane VCN 816. The customer may desire to deploy or otherwise use resources in the data plane VCN 818 that are provisioned in the control plane VCN 816, and the data plane mirror app tier 840 can facilitate the desired deployment, or other usage of resources, of the customer.

In some embodiments, the customer of the IaaS provider can apply filters to the data plane VCN 818. In this embodiment, the customer can determine what the data plane VCN 818 can access, and the customer may restrict access to public Internet 854 from the data plane VCN 818. The IaaS provider may not be able to apply filters or otherwise control access of the data plane VCN 818 to any outside networks or databases. Applying filters and controls by the customer onto the data plane VCN 818, contained in the customer tenancy 821, can help isolate the data plane VCN 818 from other customers and from public Internet 854.

In some embodiments, cloud services 856 can be called by the service gateway 836 to access services that may not exist on public Internet 854, on the control plane VCN 816, or on the data plane VCN 818. The connection between cloud services 856 and the control plane VCN 816 or the data plane VCN 818 may not be live or continuous. Cloud services 856 may exist on a different network owned or operated by the IaaS provider. Cloud services 856 may be configured to receive calls from the service gateway 836 and may be configured to not receive calls from public Internet 854. Some cloud services 856 may be isolated from other cloud services 856, and the control plane VCN 816 may be isolated from cloud services 856 that may not be in the same region as the control plane VCN 816. For example, the control plane VCN 816 may be located in “Region 1,” and cloud service “Deployment 7,” may be located in Region 1 and in “Region 2.” If a call to Deployment 7 is made by the service gateway 836 contained in the control plane VCN 816 located in Region 1, the call may be transmitted to Deployment 7 in Region 1. In this example, the control plane VCN 816, or Deployment 7 in Region 1, may not be communicatively coupled to, or otherwise in communication with, Deployment 7 in Region 2.

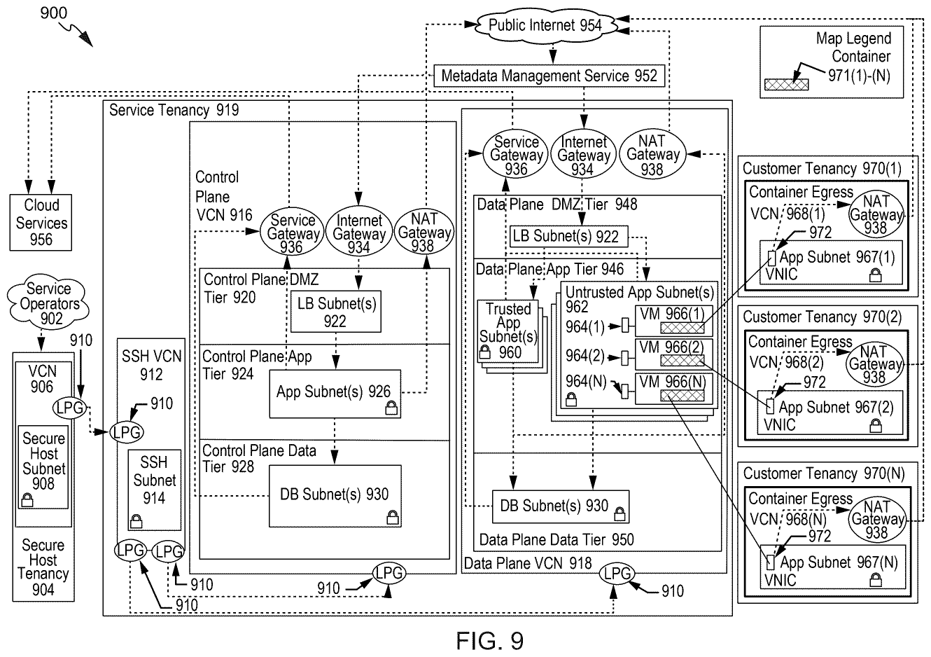

FIG. 9 is a block diagram 900 illustrating another example pattern of an IaaS architecture, according to at least one embodiment. Service operators 902 (e.g., service operators 702 of FIG. 7) can be communicatively coupled to a secure host tenancy 904 (e.g., the secure host tenancy 704 of FIG. 7) that can include a virtual cloud network (VCN) 906 (e.g., the VCN 706 of FIG. 7) and a secure host subnet 908 (e.g., the secure host subnet 708 of FIG. 7). The VCN 906 can include an LPG 910 (e.g., the LPG 710 of FIG. 7) that can be communicatively coupled to an SSH VCN 912 (e.g., the SSH VCN 712 of FIG. 7) via an LPG 910 contained in the SSH VCN 912. The SSH VCN 912 can include an SSH subnet 914 (e.g., the SSH subnet 714 of FIG. 7), and the SSH VCN 912 can be communicatively coupled to a control plane VCN 916 (e.g., the control plane VCN 716 of FIG. 7) via an LPG 910 contained in the control plane VCN 916 and to a data plane VCN 918 (e.g., the data plane 718 of FIG. 7) via an LPG 910 contained in the data plane VCN 918. The control plane VCN 916 and the data plane VCN 918 can be contained in a service tenancy 919 (e.g., the service tenancy 719 of FIG. 7).

The control plane VCN 916 can include a control plane DMZ tier 920 (e.g., the control plane DMZ tier 720 of FIG. 7) that can include load balancer (LB) subnet(s) 922 (e.g., LB subnet(s) 722 of FIG. 7), a control plane app tier 924 (e.g., the control plane app tier 724 of FIG. 7) that can include app subnet(s) 926 (e.g., similar to app subnet(s) 726 of FIG. 7), a control plane data tier 928 (e.g., the control plane data tier 728 of FIG. 7) that can include DB subnet(s) 930. The LB subnet(s) 922 contained in the control plane DMZ tier 920 can be communicatively coupled to the app subnet(s) 926 contained in the control plane app tier 924 and to an Internet gateway 934 (e.g., the Internet gateway 734 of FIG. 7) that can be contained in the control plane VCN 916, and the app subnet(s) 926 can be communicatively coupled to the DB subnet(s) 930 contained in the control plane data tier 928 and to a service gateway 936 (e.g., the service gateway of FIG. 7) and a network address translation (NAT) gateway 938 (e.g., the NAT gateway 738 of FIG. 7). The control plane VCN 916 can include the service gateway 936 and the NAT gateway 938.

The data plane VCN 918 can include a data plane app tier 946 (e.g., the data plane app tier 746 of FIG. 7), a data plane DMZ tier 948 (e.g., the data plane DMZ tier 748 of FIG. 7), and a data plane data tier 950 (e.g., the data plane data tier 750 of FIG. 7). The data plane DMZ tier 948 can include LB subnet(s) 922 that can be communicatively coupled to trusted app subnet(s) 960 and untrusted app subnet(s) 962 of the data plane app tier 946 and the Internet gateway 934 contained in the data plane VCN 918. The trusted app subnet(s) 960 can be communicatively coupled to the service gateway 936 contained in the data plane VCN 918, the NAT gateway 938 contained in the data plane VCN 918, and DB subnet(s) 930 contained in the data plane data tier 950. The untrusted app subnet(s) 962 can be communicatively coupled to the service gateway 936 contained in the data plane VCN 918 and DB subnet(s) 930 contained in the data plane data tier 950. The data plane data tier 950 can include DB subnet(s) 930 that can be communicatively coupled to the service gateway 936 contained in the data plane VCN 918.

The untrusted app subnet(s) 962 can include one or more primary VNICs 964(1)-(N) that can be communicatively coupled to tenant virtual machines (VMs) 966(1)-(N). Each tenant VM 966(1)-(N) can be communicatively coupled to a respective app subnet 967(1)-(N) that can be contained in respective container egress VCNs 968(1)-(N) that can be contained in respective customer tenancies 970(1)-(N). Respective secondary VNICs 972(1)-(N) can facilitate communication between the untrusted app subnet(s) 962 contained in the data plane VCN 918 and the app subnet contained in the container egress VCNs 968(1)-(N). Each container egress VCNs 968(1)-(N) can include a NAT gateway 938 that can be communicatively coupled to public Internet 954 (e.g., public Internet 754 of FIG. 7).

The Internet gateway 934 contained in the control plane VCN 916 and contained in the data plane VCN 918 can be communicatively coupled to a metadata management service 952 (e.g., the metadata management system 752 of FIG. 7) that can be communicatively coupled to public Internet 954. Public Internet 954 can be communicatively coupled to the NAT gateway 938 contained in the control plane VCN 916 and contained in the data plane VCN 918. The service gateway 936 contained in the control plane VCN 916 and contained in the data plane VCN 918 can be communicatively coupled to cloud services 956.

In some embodiments, the data plane VCN 918 can be integrated with customer tenancies 970. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 946. Code to run the function may be executed in the VMs 966(1)-(N), and the code may not be configured to run anywhere else on the data plane VCN 918. Each VM 966(1)-(N) may be connected to one customer tenancy 970. Respective containers 971(1)-(N) contained in the VMs 966(1)-(N) may be configured to run the code. In this case, there can be a dual isolation (e.g., the containers 971(1)-(N) running code, where the containers 971(1)-(N) may be contained in at least the VM 966(1)-(N) that are contained in the untrusted app subnet(s) 962), which may help prevent incorrect or otherwise undesirable code from damaging the network of the IaaS provider or from damaging a network of a different customer. The containers 971(1)-(N) may be communicatively coupled to the customer tenancy 970 and may be configured to transmit or receive data from the customer tenancy 970. The containers 971(1)-(N) may not be configured to transmit or receive data from any other entity in the data plane VCN 918. Upon completion of running the code, the IaaS provider may kill or otherwise dispose of the containers 971(1)-(N).

In some embodiments, the trusted app subnet(s) 960 may run code that may be owned or operated by the IaaS provider. In this embodiment, the trusted app subnet(s) 960 may be communicatively coupled to the DB subnet(s) 930 and be configured to execute CRUD operations in the DB subnet(s) 930. The untrusted app subnet(s) 962 may be communicatively coupled to the DB subnet(s) 930, but in this embodiment, the untrusted app subnet(s) may be configured to execute read operations in the DB subnet(s) 930. The containers 971(1)-(N) that can be contained in the VM 966(1)-(N) of each customer and that may run code from the customer may not be communicatively coupled with the DB subnet(s) 930.

In other embodiments, the control plane VCN 916 and the data plane VCN 918 may not be directly communicatively coupled. In this embodiment, there may be no direct communication between the control plane VCN 916 and the data plane VCN 918. However, communication can occur indirectly through at least one method. An LPG 910 may be established by the IaaS provider that can facilitate communication between the control plane VCN 916 and the data plane VCN 918. In another example, the control plane VCN 916 or the data plane VCN 918 can make a call to cloud services 956 via the service gateway 936. For example, a call to cloud services 956 from the control plane VCN 916 can include a request for a service that can communicate with the data plane VCN 918.

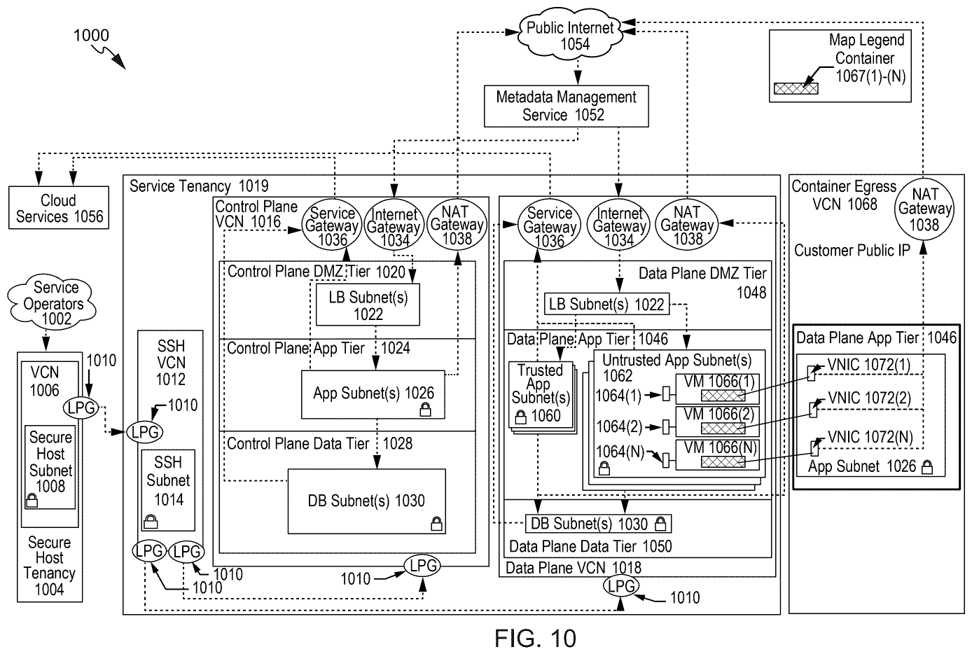

FIG. 10 is a block diagram 1000 illustrating another example pattern of an IaaS architecture, according to at least one embodiment. Service operators 1002 (e.g., service operators 702 of FIG. 7) can be communicatively coupled to a secure host tenancy 1004 (e.g., the secure host tenancy 704 of FIG. 7) that can include a virtual cloud network (VCN) 1006 (e.g., the VCN 706 of FIG. 7) and a secure host subnet 1008 (e.g., the secure host subnet 708 of FIG. 7). The VCN 1006 can include an LPG 1010 (e.g., the LPG 710 of FIG. 7) that can be communicatively coupled to an SSH VCN 1012 (e.g., the SSH VCN 712 of FIG. 7) via an LPG 1010 contained in the SSH VCN 1012. The SSH VCN 1012 can include an SSH subnet 1014 (e.g., the SSH subnet 714 of FIG. 7), and the SSH VCN 1012 can be communicatively coupled to a control plane VCN 1016 (e.g., the control plane VCN 716 of FIG. 7) via an LPG 1010 contained in the control plane VCN 1016 and to a data plane VCN 1018 (e.g., the data plane 718 of FIG. 7) via an LPG 1010 contained in the data plane VCN 1018. The control plane VCN 1016 and the data plane VCN 1018 can be contained in a service tenancy 1019 (e.g., the service tenancy 719 of FIG. 7).

The control plane VCN 1016 can include a control plane DMZ tier 1020 (e.g., the control plane DMZ tier 720 of FIG. 7) that can include LB subnet(s) 1022 (e.g., LB subnet(s) 722 of FIG. 7), a control plane app tier 1024 (e.g., the control plane app tier 724 of FIG. 7) that can include app subnet(s) 1026 (e.g., app subnet(s) 726 of FIG. 7), a control plane data tier 1028 (e.g., the control plane data tier 728 of FIG. 7) that can include DB subnet(s) 1030 (e.g., DB subnet(s) 930 of FIG. 9). The LB subnet(s) 1022 contained in the control plane DMZ tier 1020 can be communicatively coupled to the app subnet(s) 1026 contained in the control plane app tier 1024 and to an Internet gateway 1034 (e.g., the Internet gateway 734 of FIG. 7) that can be contained in the control plane VCN 1016, and the app subnet(s) 1026 can be communicatively coupled to the DB subnet(s) 1030 contained in the control plane data tier 1028 and to a service gateway 1036 (e.g., the service gateway of FIG. 7) and a network address translation (NAT) gateway 1038 (e.g., the NAT gateway 738 of FIG. 7). The control plane VCN 1016 can include the service gateway 1036 and the NAT gateway 1038.

The data plane VCN 1018 can include a data plane app tier 1046 (e.g., the data plane app tier 746 of FIG. 7), a data plane DMZ tier 1048 (e.g., the data plane DMZ tier 748 of FIG. 7), and a data plane data tier 1050 (e.g., the data plane data tier 750 of FIG. 7). The data plane DMZ tier 1048 can include LB subnet(s) 1022 that can be communicatively coupled to trusted app subnet(s) 1060 (e.g., trusted app subnet(s) 960 of FIG. 9) and untrusted app subnet(s) 1062 (e.g., untrusted app subnet(s) 962 of FIG. 9) of the data plane app tier 1046 and the Internet gateway 1034 contained in the data plane VCN 1018. The trusted app subnet(s) 1060 can be communicatively coupled to the service gateway 1036 contained in the data plane VCN 1018, the NAT gateway 1038 contained in the data plane VCN 1018, and DB subnet(s) 1030 contained in the data plane data tier 1050. The untrusted app subnet(s) 1062 can be communicatively coupled to the service gateway 1036 contained in the data plane VCN 1018 and DB subnet(s) 1030 contained in the data plane data tier 1050. The data plane data tier 1050 can include DB subnet(s) 1030 that can be communicatively coupled to the service gateway 1036 contained in the data plane VCN 1018.

The untrusted app subnet(s) 1062 can include primary VNICs 1064(1)-(N) that can be communicatively coupled to tenant virtual machines (VMs) 1066(1)-(N) residing within the untrusted app subnet(s) 1062. Each tenant VM 1066(1)-(N) can run code in a respective container 1067(1)-(N), and be communicatively coupled to an app subnet 1026 that can be contained in a data plane app tier 1046 that can be contained in a container egress VCN 1068. Respective secondary VNICs 1072(1)-(N) can facilitate communication between the untrusted app subnet(s) 1062 contained in the data plane VCN 1018 and the app subnet contained in the container egress VCN 1068. The container egress VCN can include a NAT gateway 1038 that can be communicatively coupled to public Internet 1054 (e.g., public Internet 754 of FIG. 7).

The Internet gateway 1034 contained in the control plane VCN 1016 and contained in the data plane VCN 1018 can be communicatively coupled to a metadata management service 1052 (e.g., the metadata management system 752 of FIG. 7) that can be communicatively coupled to public Internet 1054. Public Internet 1054 can be communicatively coupled to the NAT gateway 1038 contained in the control plane VCN 1016 and contained in the data plane VCN 1018. The service gateway 1036 contained in the control plane VCN 1016 and contained in the data plane VCN 1018 can be communicatively coupled to cloud services 1056.

In some examples, the pattern illustrated by the architecture of block diagram 1000 of FIG. 10 may be considered an exception to the pattern illustrated by the architecture of block diagram 900 of FIG. 9 and may be desirable for a customer of the IaaS provider if the IaaS provider cannot directly communicate with the customer (e.g., a disconnected region). The respective containers 1067(1)-(N) that are contained in the VMs 1066(1)-(N) for each customer can be accessed in real-time by the customer. The containers 1067(1)-(N) may be configured to make calls to respective secondary VNICs 1072(1)-(N) contained in app subnet(s) 1026 of the data plane app tier 1046 that can be contained in the container egress VCN 1068. The secondary VNICs 1072(1)-(N) can transmit the calls to the NAT gateway 1038 that may transmit the calls to public Internet 1054. In this example, the containers 1067(1)-(N) that can be accessed in real-time by the customer can be isolated from the control plane VCN 1016 and can be isolated from other entities contained in the data plane VCN 1018. The containers 1067(1)-(N) may also be isolated from resources from other customers.

In other examples, the customer can use the containers 1067(1)-(N) to call cloud services 1056. In this example, the customer may run code in the containers 1067(1)-(N) that requests a service from cloud services 1056. The containers 1067(1)-(N) can transmit this request to the secondary VNICs 1072(1)-(N) that can transmit the request to the NAT gateway that can transmit the request to public Internet 1054. Public Internet 1054 can transmit the request to LB subnet(s) 1022 contained in the control plane VCN 1016 via the Internet gateway 1034. In response to determining the request is valid, the LB subnet(s) can transmit the request to app subnet(s) 1026 that can transmit the request to cloud services 1056 via the service gateway 1036.

It should be appreciated that IaaS architectures 700, 800, 900, 1000 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

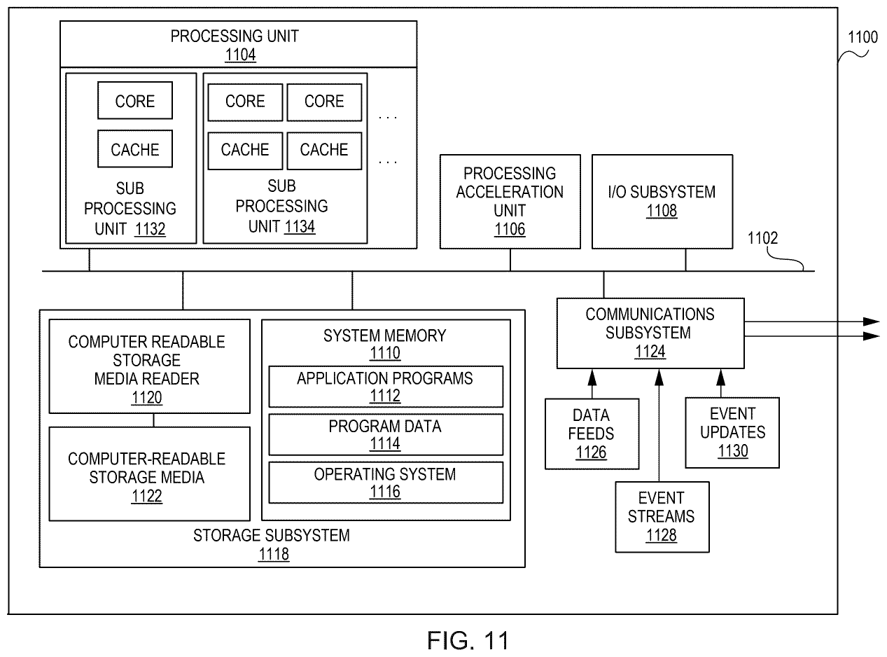

FIG. 11 illustrates an example computer system 1100, in which various embodiments may be implemented. The system 1100 may be used to implement any of the computer systems described above. As shown in the figure, computer system 1100 includes a processing unit 1104 that communicates with a number of peripheral subsystems via a bus subsystem 1102. These peripheral subsystems may include a processing acceleration unit 1106, an I/O subsystem 1108, a storage subsystem 1118 and a communications subsystem 1124. Storage subsystem 1118 includes tangible computer-readable storage media 1122 and a system memory 1110.

Bus subsystem 1102 provides a mechanism for letting the various components and subsystems of computer system 1100 communicate with each other as intended. Although bus subsystem 1102 is shown schematically as a single bus, alternative embodiments of the bus subsystem may utilize multiple buses. Bus subsystem 1102 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1104, which can be implemented as one or more integrated circuits (e.g., a conventional microprocessor or microcontroller), controls the operation of computer system 1100. One or more processors may be included in processing unit 1104. These processors may include single core or multicore processors. In certain embodiments, processing unit 1104 may be implemented as one or more independent processing units 1132 and/or 1134 with single or multicore processors included in each processing unit. In other embodiments, processing unit 1104 may also be implemented as a quad-core processing unit formed by integrating two dual-core processors into a single chip.

In various embodiments, processing unit 1104 can execute a variety of programs in response to program code and can maintain multiple concurrently executing programs or processes. At any given time, some or all of the program code to be executed can be resident in processor(s) 1104 and/or in storage subsystem 1118. Through suitable programming, processor(s) 1104 can provide various functionalities described above. Computer system 1100 may additionally include a processing acceleration unit 1106, which can include a digital signal processor (DSP), a special-purpose processor, and/or the like.

I/O subsystem 1108 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1100 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1100 may comprise a storage subsystem 1118 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1104 provide the functionality described above. Storage subsystem 1118 may also provide a repository for storing data used in accordance with the present disclosure.

As depicted in the example in FIG. 11, storage subsystem 1118 can include various components including a system memory 1110, computer-readable storage media 1122, and a computer readable storage media reader 1120. System memory 1110 may store program instructions that are loadable and executable by processing unit 1104. System memory 1110 may also store data that is used during the execution of the instructions and/or data that is generated during the execution of the program instructions. Various different kinds of programs may be loaded into system memory 1110 including but not limited to client applications, Web browsers, mid-tier applications, relational database management systems (RDBMS), virtual machines, containers, etc.

System memory 1110 may also store an operating system 1116. Examples of operating system 1116 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1100 executes one or more virtual machines, the virtual machines along with their guest operating systems (GOSs) may be loaded into system memory 1110 and executed by one or more processors or cores of processing unit 1104.

System memory 1110 can come in different configurations depending upon the type of computer system 1100. For example, system memory 1110 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1110 may include a basic input/output system (BIOS) containing basic routines that help to transfer information between elements within computer system 1100, such as during start-up.

Computer-readable storage media 1122 may represent remote, local, fixed, and/or removable storage devices plus storage media for temporarily and/or more permanently containing, storing, computer-readable information for use by computer system 1100 including instructions executable by processing unit 1104 of computer system 1100.

Computer-readable storage media 1122 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1122 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1122 may include, but is not limited to, Zip® drives, flash memory cards, universal serial bus (USB) flash drives, secure digital (SD) cards, DVD disks, digital video tape, and the like. Computer-readable storage media 1122 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1100.

Machine-readable instructions executable by one or more processors or cores of processing unit 1104 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1124 provides an interface to other computer systems and networks. Communications subsystem 1124 serves as an interface for receiving data from and transmitting data to other systems from computer system 1100. For example, communications subsystem 1124 may enable computer system 1100 to connect to one or more devices via the Internet. In some embodiments communications subsystem 1124 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof)), global positioning system (GPS) receiver components, and/or other components. In some embodiments communications subsystem 1124 can provide wired network connectivity (e.g., Ethernet) in addition to or instead of a wireless interface.

In some embodiments, communications subsystem 1124 may also receive input communication in the form of structured and/or unstructured data feeds 1126, event streams 1128, event updates 1130, and the like on behalf of one or more users who may use computer system 1100.

By way of example, communications subsystem 1124 may be configured to receive data feeds 1126 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1124 may also be configured to receive data in the form of continuous data streams, which may include event streams 1128 of real-time events and/or event updates 1130, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1124 may also be configured to output the structured and/or unstructured data feeds 1126, event streams 1128, event updates 1130, and the like to one or more databases that may be in communication with one or more streaming data source computers coupled to computer system 1100.

Computer system 1100 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1100 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method comprising:
receiving, at an application programming interface endpoint, a request comprising an unstructured text query;
generating, using a semantic embedding model and based on the unstructured text query, a query vector representing a semantic meaning of the unstructured text query;
executing, using the query vector, a hybrid search against a pre-indexed vector database, wherein:
the pre-indexed vector database comprises vector representations for medical codings and semantic object schemas, and
the hybrid search combines a vector similarity search with a keyword search to retrieve a list of most relevant candidate codings and a list of most relevant candidate schemas; and
providing, via the application programming interface, a structured response comprising a ranked list of the most relevant candidate codings and a ranked list of the most relevant candidate schemas.

2. The computer-implemented method of claim 1, further comprising prior to generating the query vector, preprocessing the unstructured text query by performing key phrase extraction to identify one or more clinical terms in the unstructured text query, wherein the query vector is generated using the semantic embedding model and based on the one or more clinical terms.

3. The computer-implemented method of claim 1, wherein the vector similarity search is a k-Nearest Neighbors (kNN) vector similarity search that is executed based on the query vector to identify a first set of results, the keyword search is executed based on the query vector to identify a second set of results, and the first and second sets of results are combined based on a weight for the vector similarity search and a weight for the key word search to retrieve the list of most relevant candidate codings and the list of most relevant candidate schemas.

4. The computer-implemented method of claim 1, wherein the structured response is configured for consumption by a downstream schema synthesizer configured to construct a machine-readable representation of the unstructured text query.

5. The computer-implemented method of claim 1, wherein the request further comprises a search configuration, and wherein executing the hybrid search is governed by one or more parameters in the search configuration, the one or more parameters comprising a search size, a score threshold, a weight for the vector similarity search, a weight for the key word search, or any combination thereof.

6. The computer-implemented method of claim 1, further comprising:
generating, by one or more generative models based on the unstructured text query and the structured response, an execution plan that identifies one or more agents to execute one or more actions or operations responsive to the unstructured text query and the structured response;
executing, by the one or more agents, the one or more actions or operations on one or more assets and/or a context and memory store, wherein the execution of the one or more actions or operations generates output data from the one or more assets and/or relevant context and memory information from the context and memory store;
generating, by one or more generative models based on an output prompt, a response to the unstructured text query, wherein the output prompt comprises the output data and/or the relevant context and the memory information; and
providing the response to the unstructured text query.

7. The computer-implemented method of claim 1, further comprising:
generating, by one or more generative models based on an output prompt, a response to the unstructured text query, wherein the output prompt comprises the unstructured text query and/or the ranked list of the most relevant candidate codings and the ranked list of the most relevant candidate schemas; and
providing the response to the unstructured text query.

8. A computer-implemented method comprising:
receiving, at an application programming interface endpoint, a structured request comprising one or more pre-identified text entities, each text entity of the one or more pre-identified text entities having an associated entity type;
for each text entity, generating, using a semantic embedding model and based on the structured request, a query vector representing a semantic meaning of each entity;
executing, using the query vector for each text entity, a hybrid search for each entity against a pre-indexed vector database, wherein:
the pre-indexed vector database comprises vector representations for medical codings,
the hybrid search combines a vector similarity search with a keyword search to retrieve a list of most relevant candidate codings and corresponding confidence scores, and
the hybrid search is constrained by the entity type associated with the text entity; and
providing, via the application programming interface, a structured response that associates each of the text entities with a ranked list of matched concepts based on the list of most relevant candidate codings and the corresponding confidence scores.

9. The computer-implemented method of claim 8, wherein the vector similarity search is a k-Nearest Neighbors (kNN) vector similarity search that is executed based on the query vector to identify a first set of results, the keyword search is executed based on the query vector to identify a second set of results, and the first and second sets of results are combined based on a weight for the vector similarity search and a weight for the key word search to retrieve the list of most relevant candidate codings.

10. The computer-implemented method of claim 8, wherein the one or more identified text entities in the structured request are generated by a Named Entity Recognition (NER) process applied to a natural language utterance.

11. The computer-implemented method of claim 8, wherein constraining the hybrid search based on the entity type associated with the text entity comprises searching only a subset of the pre-indexed vector database that corresponds to concepts associated with the entity type.

12. The computer-implemented method of claim 8, wherein the structured request further comprises a list of one or more target coding systems, and wherein the hybrid search for each text entity is further constrained to concepts belonging to the one or more target coding systems.

13. The computer-implemented method of claim 8, further comprising:
generating, by one or more generative models based on a natural language utterance, an execution plan that identifies one or more agents to execute one or more actions or operations responsive to the natural language utterance;
executing, by the one or more agents, the one or more actions or operations on one or more assets and/or a context and memory store, wherein the execution of the one or more actions or operations generates output data from the one or more assets and/or relevant context and memory information from the context and memory store;
generating, by one or more generative models based on an output prompt, a response to the natural language utterance, wherein the output prompt comprises: (i) the output data and/or the relevant context and the memory information, and (ii) the structured response that associates each of the text entities with the ranked list of matched concepts; and
providing the response to the natural language utterance.

14. The computer-implemented method of claim 8, further comprising:
generating, by one or more generative models based on an output prompt, a response to a natural language utterance, wherein the output prompt comprises: (i) the natural language utterance, and (ii) the structured response that associates each of the text entities with a ranked list of matched concepts; and
providing the response to the natural language utterance.

15. The computer-implemented method of claim 8, further comprising:
searching, using the structured response that associates each of the text entities with the ranked list of matched concepts, a semantic knowledge graph to identify one or more relationships between text entities;
generating, by one or more generative models based on an output prompt, a response to a natural language utterance, wherein the output prompt comprises: (i) the natural language utterance, (ii) the structured response that associates each of the text entities with the ranked list of matched concepts, and the (iii) the one or more relationships between the text entities; and
providing the response to the natural language utterance.

16. A system comprising:
one or more processing systems; and
one or more computer-readable media storing instructions which, when executed by the one or more processing systems, cause the system to perform operations comprising:
receiving, at an application programming interface endpoint, a request comprising an unstructured text query or one or more pre-identified text entities;
generating, using a semantic embedding model and based on the request, a query vector representing a semantic meaning of the unstructured text query or the one or more pre-identified text entities;
executing, using the query vector, a hybrid search against a pre-indexed vector database, wherein:
the pre-indexed vector database comprises vector representations for medical codings and semantic object schemas, and
the hybrid search combines a vector similarity search with a keyword search to retrieve a list of most relevant candidate codings, a list of most relevant candidate schemas, or both; and
providing, via the application programming interface, a structured response comprising: (i) a ranked list of the most relevant candidate codings and a ranked list of the most relevant candidate schemas, or (ii) a ranked list of matched concepts based on the list of most relevant candidate codings.

17. The system of claim 16, wherein the vector similarity search is a k-Nearest Neighbors (kNN) vector similarity search that is executed based on the query vector to identify a first set of results, the keyword search is executed based on the query vector to identify a second set of results, and the first and second sets of results are combined based on a weight for the vector similarity search and a weight for the key word search to retrieve the list of most relevant candidate codings, the list of most relevant candidate schemas, or both.

18. The system of claim 16, wherein the request further comprises a search configuration, and wherein executing the hybrid search is governed by one or more parameters in the search configuration, the one or more parameters comprising a search size, a score threshold, a weight for the vector similarity search, a weight for the key word search, or any combination thereof.

19. The system of claim 16, wherein the operations further comprise:
generating, by one or more generative models based on a natural language utterance, the ranked list of the most relevant candidate codings, and the ranked list of the most relevant candidate schemas, an execution plan that identifies one or more agents to execute one or more actions or operations responsive to the natural language utterance;
executing, by the one or more agents, the one or more actions or operations on one or more assets and/or a context and memory store, wherein the execution of the one or more actions or operations generates output data from the one or more assets and/or relevant context and memory information from the context and memory store;
generating, by one or more generative models based on an output prompt, a response to the natural language utterance, wherein the output prompt comprises the output data and/or the relevant context and the memory information; and
providing the response to the natural language utterance.

20. The system of claim 16, wherein the operations further comprise:
generating, by one or more generative models based on an output prompt, a response to a natural language utterance, wherein the output prompt comprises: (i) the natural language utterance, and (ii) the structured response that associates each of the text entities with a ranked list of matched concepts; and
providing the response to the natural language utterance.
